(19) United States
(12) Patent Application Publication
(10) Pub. No.: US 2025/0367041 A1
(43) Pub. Date: Dec. 4, 2025

(54) Advanced High-Tech Flushable Diaper Device and System

(71) Applicant: Guy Stanley Swanson, Bonham, TX (US)

(72) Inventor: Guy Stanley Swanson, Bonham, TX (US)

(21) Appl. No.: 18/732,145

(22) Filed: Jun. 3, 2024

(51) Int. Cl. A61F 13/495; A61F 5/44; A61F 5/442; A61F 7/00; A61F 7/02; A61F 13/42; A61F 13/84

(52) U.S. Cl. CPC A61F 13/495; A61F 5/4405; A61F 5/442; A61F 7/02; A61F 2007/0059; A61F 2007/0071; A61F 2007/0236; A61F 2013/424; A61F 2013/8494

(57) ABSTRACT

This invention is an advanced, high-tech, flushable, diaper system that would eliminate the need for parents and caretakers to change diapers in the traditional way, whereby users need to have urine and feces wiped clean from their bodies and used diapers thrown away to be added to landfills or, in cases of reusable cloth diapers, washed at home or sent off to be washed and sanitized professionally. It would improve comfort and cleanliness for many and provide more dignity for the incontinent and bedridden. It would benefit those on long work shifts without bathroom breaks like pilots, astronauts, and surgeons. It would be useful to those in remote locations like distant travelers, skiers, hikers, mountain climbers, and deep sea divers or in harsh environments, where a toilet is not easily accessible like on the battlefield. Versions of this advanced diaper system may also be used for certain animals and pets.

### BACKGROUND

### Field of the Invention

This invention broadly relates to the field of hygienic fecal and urine management. More specifically, this invention relates to a diaper system that effectively serves as a fecal and urine capture, collection, storage, and disposal device and method.

### The Prior Art

Throughout history, bodily waste management systems have been designed and built, giving us modern-day toilets, diapers, absorbent pads, urine receptacles, and bedpans. A great deal of time, effort, and thought has gone into trying to solve problems created by bodily waste, namely in the area of comfort, sanitation, and personal hygiene. These are problems nurses and caretakers face when caring for bedridden patients, the sick, the handicapped, the mentally impaired, newborn babies, and those with incontinence—a loss of bladder or bowel control, varying from a slight to complete inability to control one's own urination or defecation. They include problems faced by our astronauts in space who are forced to deal with high, low, or zero gravity as well as pilots on long flights and those whose work or activities require long sessions. A really good solution seems to be elusive.

Some of the waste capture and/or storage devices have evolved over time but still fall short of a satisfactory solution, while others have barely changed at all. Early on, babies were wrapped in reusable cloth diapers made of cotton or wool that were not ideal for containing wet, smelly waste and which had to be thoroughly washed and sanitized before reusing. However, improvements were made over the years. Starting around the early 20th century, rubber baby pants could be used to place over cloth diapers to help retain bodily waste fluids and prevent them from leaking. In the 1930's diaper services became popular, whereby parents and caretakers could send off dirty diapers to be professionally cleaned. Then in the late 1940's and early 1950's, reusable plastic diaper covers were developed to be worn over cloth diapers to better prevent urine and liquid feces from leaking. As the decades rolled on, deposable diapers made of synthetic and absorbent materials were developed that were even better at containing feces and bio-fluids. They added convenience and ease for parents and caretakers but sadly have also added to the landfills at an alarming rate that is believed to be environmentally harmful. And nowadays, metal and plastic snaps as well as hook and loop patches have replaced safety pins to hold the diapers in place. Cloth diapers are currently being made from a variety of new materials such as cellulose and plush bamboo.

Unfortunately, these systems and devices are still quite primitive and crude. For instance, after being soiled and/or wetted, both reusable cloth diapers and disposable synthetic diapers have to be carefully removed from patients, babies, or other users, to be properly cleaned, managed, or disposed of. These diaper users then must be thoroughly cleaned and re-diapered. During this whole process, the risk of coming into accidental contact with urine and fecal matter, and thus dangerous pathogens, is very high. Traditional diapers leak and have to be changed often. They cause skin rashes and allow urine and feces to mix and contaminate the wearer's genitals, which can at times lead to urinary tract infections and sores. They don't do a good job of containing unpleasant, offensive odors. Disposable diapers are the most commonly used at present. Though they do add convenience and ease, they are more expensive in the long run, especially when adding up the number of disposable diapers used during the years of a baby to a young child.

Sadly, disposable diapers also add very large amounts of non-biodegradable waste and in some cases relatively slow-to-biodegrade waste to the landfills at an alarming rate, which is a growing environmental hazard and concern. Each year, there are enough disposable diapers that are thrown away that they could fill up 30 Empire State buildings!

Most disposable diapers purchased today are not biodegradable nor eco-friendly. According to the Environmental Protection Agency (EPA), waste filled plastic diapers that are potentially toxic sit buried in landfills for centuries. It's estimated that disposable diapers take about 500 years or more to completely decompose, due to all the plastic usually used to make them. In many cases, toxic materials are used. In recent years, disposable diaper companies have started providing biodegradable diapers that decompose 10 times faster than traditional disposable diapers and therefore completely decompose within about 50 years. That's good news but still very alarming and still taking way too long to degrade. The average baby goes through about 7,000 diapers before potty training even begins, and there are a lot of babies in the world. The Centers for Disease Control and Prevention (CDC) recently reported a little under 3.6 million births within the U.S. for the year 2023, making it about 10,000 births per day on average, thus disposable diapers are the 3^{rd} largest form of household waste in the United States alone. Other countries with a much higher population go through even more of these environmentally harmful disposable diapers. Thankfully, more and more environmentally conscious parents are now choosing reusable cloth diapers. Even though cloth diapers do have several drawbacks, at least the bodily waste cleaned from them goes to the sewer system instead of landfills in a hazardous way.

One last respectful criticism about traditional diapers, whether reusable or disposable, is too often the users go an hour or more before their diaper is even changed, leading to skin irritations, diaper rashes, sores, infections, and great discomfort. Given these drawbacks and the above statistics on disposable diapers, it's clear that a better diaper system and solution is needed for handling, managing, and disposing of bodily waste.

Having to use bedpans is equally problematic, often involving discomfort, accidental spills, and contamination. Bedpans, in essence, haven't changed much either. They are essentially still a wide open container, whether made of metal, wood, or plastic, placed under a user. A device that once used has to be washed out by hand, a very unpleasant experience and duty as anyone who has done it will testify. It's unsightly, allows for greater unpleasant odor diffusion, just as embarrassing as a traditional diaper and at times more so.

Bedpans are best suited for those who can predict and control their bowel movements. At times, patients are too helpless or debilitated that they can't even be moved into position to use a bedpan, either because they are in too much pain, are too ill, are unconscious, or are paralyzed.

Bedpans are not always adequately cleaned, especially in work places that are understaffed or overwhelmed with patients, and therefore pass on harmful bacteria and other microbes to other patients or equipment in a hospital or retirement home. This amplifies the health risks to hospital aids, patients, and retirement home residents.

Traditional diapers and bedpans are very messy, unsightly, and quite time consuming for parents, nurses, and caretakers to deal with. With these traditional waste capture and collection devices, feces and urine is easily transmitted to bed spreads, blankets, furniture, hospital equipment, and the floor. Some of the newly patented bedpans or bedpan type devices, such as those documented in U.S. Pat. No. 10,765,578B2 and U.S. Pat. No. 4,950,262, strive to make improvements. However, it's difficult to determine whether or not they have. After careful review, they still possess many of the problems and issues that come with basic bedpans and disposable diapers. For instance, they still appear to be difficult to use or wear and seem to be for temporary short-term use, only, before needing to be disposed of and replaced. I'm hoping to move past disposable devices. And the novel aspects of these newer devices are questionable as to whether they would actually work or function or be as effective as their inventors visualize and imagine. Using these outdated and hopefully soon to become old fashioned diapers, bedpans, and methods should become things of the past.

Urinary catheters were created to help capture and dispose of urine from patients in hospitals, nursing homes, retirement homes, and others who could not easily use a toilet, regularly. They are essentially small, thin flexible tubes that are inserted into a person's urethra and bladder to help relieve his or her urine buildup. They are painful and embarrassing to have them inserted. They're uncomfortable. They limit freedom of movement. And they only address the urinary part of bodily waste management and not the fecal matter.

Other devices have been invented and devised such as urinary wicks that use capillary action and vacuum suction like the device described in the US20230277362A1 patent, which may be the device commercially known as PureWick™ or at least one very similar. Some of the main disadvantages worth covering regarding these type of urine wicking systems is that they greatly limit movement, given that these devices require a tube to be attached during many hours of use (not just during times of urination) which itself is attached to a nearby canister. This would limit a user's freedom to freely turn and roll in bed or to easily get up from a bed or a chair to move around. Another disadvantage worth noting is that the user of a urine wicking type device would be in prolonged contact with the wetness of urine after using it once. This leads to a third disadvantage of a higher chance of urinary tract infection (UTI), given the prolonged contact with urine which can harbor bacteria. Yet, another disadvantage of this Pure Wick™ invention or one like it is that currently it's designed for women only. One final disadvantage worth covering is that it only manages urine waste and not feces, vaginal discharges, or other bodily waste.

There are other invented devices, old and new, that use either manual suction pumps, powered vacuums, differential pressure, or gravity to collect and move urine from the body, through a tube, to a bag or a storage tank or reservoir of some sort, such as the following patented devices; U.S. Pat. Nos. 3,602,923A, 4,270,539A, 6,311,339B1, and 6,904,621. Many of these newer inventions have helped to overcome some of the shortcomings and drawbacks of traditional diapers and bodily waste collection methods but still do not fully solve all or many of their disadvantages to an appreciable degree. Many of them (not all) require a great deal of assistance from a caretaker, do not prevent urine from coming into contact with a female's vagina, limit the user's freedom of movement, too invasive, difficult to clean and sanitize, and usually require electrical power.

Another newly published device that also proposes using a vacuum force and a tube to collect and transport bodily waste is the one described in the US20220379001 A1. Only it proposes it could safely remove solid fecal waste in this manner, which again is questionable. Many years ago, I too had conceived of similar ideas of a bodily waste collection device involving rectal inserts, both rigid and non-rigid, that could serve as a type of catheter for the anus to collect solid waste (though my conceptions never involved the use of vacuum pressure during defecation, only after). But I always worried that these rectal inserts would be naturally discharged or pushed out by the rectal and sphincter muscles, could cause sphincter muscle stimulus response issues (sphincter dysfunction), anal erosion, or even permanent damage over time with long term use, and would be a very unpleasant and invasive job for any parent or caretaker to always have to insert these devices into the rectums of patients or users as well as uncomfortable and embarrassing for the patients and/or users. And again, users and caretakers would have to deal with long tubes attached to the body for long hours, possibly even days, which are problematic and limit movement during use and while trying to sleep.

There have been devices patented that claim to effectively allow a user to directly urinate into a plastic disposable bag, such as the one discussed in U.S. Pat. No. 3,292,626 or directly defecate into a flexible, disposable, bag-like container, such as the one described in US20230181384, which can then be properly disposed of later. Both use adhesive material and surfaces that are intended to form an attachment and secured seal to the body of the user. Both are meant to be disposable. And both can only address one form of bodily waste, only urine or only feces, but not both urine and feces. I predict that the thin adhesive seal will not be adequate to keep the device properly attached and sealed. I think body sweat and the curved contours of the human body will cause the sticky seals to loosen and separate over time, if only in parts, and will not adequately stay in place. These thin top-surface adhesives would likely be problematic for users with a lot of body hair and difficult to remove from the body hair even though their seal along the curving contours surrounding the anus has been broken or loosened. It's also not clear how these devices can be effectively and comfortably worn by a user. I'm thinking alignment is going to be a huge problem. That is to say getting these adhesive flaps and small bag openings like those in the aforementioned US20230181384 adequately aligned with the anus is going to be largely a guessing game or mainly done by touch and feel, which would be undesirable by most and would almost require a developed skill or talent unless these bags were made of very clear and transparent materials to see through or included a clear plastic viewport on the opposite side, but are not and do not according to the description within the specification of that invention.

There has also been several attempts by the National Aeronautical Space Administration (NASA) to manage bodily waste, and the challenge to adequately deal with it in the field of space exploration continues to this day.

In a very informative Business Insider web post article, Hilary Brueck writes a brief history on NASA's waste management efforts, which I will provide a summary on below. For more details visit https://www.businessinsider.com/how-nasa-astronauts-pee-and-poop-in-space-2018-8

Ever since the early 1960's, such as when the first American astronaut Alan B. Shepard had little choice but to simply pee in his pants while sitting on top of a Redstone rocket after waiting hours to launch, NASA has struggled with the human waste problem. The most challenging was how to deal with an astronaut's need to poop, especially while in space in zero gravity. A variety of possible solutions have been tried, but by their own admission not much progress has been made in this area over the last 60 years. Most of the waste management systems NASA has come up with so far essentially involve crude devices like pee bags, rubber sleeves that looked like condoms called “roll-on cuffs,” which women can't use, plastic cups connected to vacuum tubes, metal collection storage tanks, and absorbent layered undergarments that are basically adult diapers. Early methods to deal with poop in zero gravity, such as during the Gemini and Apollo programs, involved astronauts taping plastic bags onto their rear end. At times these rubber cuffs would leak, poop would escape from the plastic bags and float away, and astronauts would have to wear gloves to pack down their own feces into storage containers.

It's important to briefly cover some of these devices and methods used by NASA in a little more detail in order to adequately compare them to the present invention and to reveal many of their flaws and limitations.

In my view, the waste management system NASA had worked out was quite time-consuming, extremely unpleasant, and overall rather difficult and problematic. NASA goes into great detail on the designs and operations of the main waste management systems used during the early manned space program in Chapter 2 of an article titled, SP-368 Biomedical Results of Apollo by Richard L. Sauer of the Lyndon B. Johnson Space Center and George K. Jorgensen from The Boeing Company. https://www.hq.nasa.gov/pao/History/SP-368/s6ch2.htm

As Richard L. Sauer and George K. Jorgensen write in Chapter 2 Waste Management System, “Defecation and urination have been bothersome aspects of space travel from the beginning of manned space flight.” In their introduction they explain that due to the weightless environment of space and the size and weight limitations of NASA's spacecraft, it was a very difficult problem to solve. These limitations led to the waste management systems described below.

As already briefly mentioned, one of the earliest devices created to strictly capture and collect urine for astronauts during manned space flight missions was the urine transfer system (UTS). It consisted of a latex roll-on cuff or rubber tube that functioned as an external catheter for males only. It connected to a plastic tube with a valve and clamp as well as a urine collection bag with a capacity of about 1200 ml. The roll-on cuffs had to be replaced after 5 to 6 urinations and at times they leaked.

Another similar device and urinary system was the urine collection and transfer assembly (UCTA), which was designed to accommodate an astronaut while wearing a pressure suit. The urine collection and transfer assembly also had a rubber roll-on cuff. But this device had a collection bladder worn around the waist that could be drained either while an astronaut was in his suit or after having removed it by connecting the urine transfer hose to the spacecraft's waste management panel.

A newer device that served as an upgrade from the UTS and less intimate, in terms of direct contact with one's genitals, was the urine receptacle assembly (URA). It was basically a hand-held metal cylinder with a honeycombed insert connected to a flexible urine dump hose line, which was connected to the waste management panel. A lid could be put on when not in use to block out cabin airflow and to allow the vacuum of space to vent out and “dump” the urine.

For capturing and collecting feces, NASA engineers developed and patented a fecal collection device called a relief container, which is described in U.S. Pat. No. 3,421,506. Later called the fecal collection assembly, Sauer and others explain, “The device used was a plastic bag . . . with a flange at the opening . . . which was taped to the buttocks to capture feces.” A “finger cot was employed to position the fecal bag over the anus. The finger cot was also used after defecation to separate fecal matter from the anal area and push it to the bottom of the bag. The bag was then removed from the buttocks, and the anus was cleaned with tissue wipes. These were disposed of into the fecal bag.” At this point, “the crewmember was required to seal the bag and knead it in order to mix a liquid bactericide with the [fecal matter] contents to provide the desired degree of feces stabilization.” Then the fecal bag “was rolled into the smallest possible volume and then placed in the waste stowage compartment.”

Astronaut Bill Anders who flew with Frank Borman and Jim Lovell around the Moon during the Apollo 8 mission in 1968 reported that he never had to defecate into a bag during their trip. He chose to hold it for nearly a week and just barely made it to a toilet in time upon his return to Earth. To him, the idea of using NASA's fecal bag method was just too gruesome and unpleasant.

When the astronauts were suited up in their spacesuits, the fecal containment system was used. This was essentially a diaper, a pair of underwear with multiple layers of absorbent material, which later had to be carefully removed and bagged for onboard storage until their return.

The report goes on to express the sentiments of the crewmen, “the system must be given poor marks. The principal problem with both the urine and fecal collection systems was the fact that these required more manipulation than crewmen were used to in the Earth environment and were, as a consequence, found to be objectionable. The urine receptacle assembly represented an attempt to preclude crew handling of urine specimens but, because urine spills were frequent, the objective of “sanitizing” the process was thwarted. The fecal collection system presented an even more distasteful set of problems. The collection process required a great deal of skill to preclude escape of feces from the collection bag and consequent soiling of the crew, their clothing, or cabin surfaces. The fecal collection process was, moreover, extremely time consuming because of the level of difficulty involved with use of the system. An Apollo 7 astronaut estimated the time required to correctly accomplish the process at 45 minutes.”

The Chapter 2 article closes with the following comments. “Attempts to improve the fecal collection system, as exemplified by the modified fecal/emesis collection bags flown on Apollo 16, failed in the crew's estimation . . . . In addition to being marginal from a hygienic standpoint, use of the collection devices required many steps and the expenditure of a considerable amount of time. The problem of odor was continually present because of the lack of a positive means of eliminating defecation odors. The Apollo waste management system's design and operations pointed to the need for several improvements in future missions.”

Many prior devices, systems, and methods were and still are intended for either strictly males or strictly females but not both or for strictly urine collecting and containment or feces collecting and containment, to address urinary incontinence only or bowel incontinence only but rarely ever a solution for managing both, simultaneously. And these current devices and solutions are usually designed for humans only and do not include animals or vice versa. So in my view, there is a great need for an improved bodily waste management system. In particular and especially, a new diaper system. One that would address all the problems I've discussed so far and more, an all-in-one solution.

### SUMMARY OF THE INVENTION

This invention serves as a reusable, flushable, cleanable diaper for babies, the elderly, the incontinent, the hospitalized, the handicapped, the mentally challenged, or incapacitated, and for those who have long work sessions or are in remote locations, where toilets are not available. This invention will greatly improve personal hygiene for diaper users by greatly reducing the level of contact that is made between their body and their waste, such as feces, urine, and menstrual blood. A less invasive and at times non-invasive method for managing bodily waste from both humans and animals. This advanced, novel, diaper system and method is carefully described and claimed in great detail in the following paragraphs.

### The Objects and Advantages of the Invention

I believe the advanced high-tech diaper device and system I am proposing is an all-in-one solution. It's a diaper system designed to capture, collect, contain, self-clean, and easily dispose of urine and stool in a more sanitary way. Below are the objects as well as the advantages of the present invention

### The Objects of the Invention

A primary object of this invention is to provide an improved bodily waste collection and storage diaper device for both males and females, young and old, sick and well, mobile and immobile, and for humans and animals.

Another leading object of this invention is to provide a sophisticated diaper device that can be easily cleaned by flushing water and other cleaning and sanitizing fluids throughout by means of its own built-in flushing system.

Another object is to create a bodily waste (urine, feces, menstrual blood, vaginal discharge, etc.) capture and collection device that can be worn by a user.

Another object is to make an advanced diaper device and system that is portable.

A fifth object of this invention is to provide a device that can be used as a flushable bedpan, whereby bodily waste is captured, collected, and even stored when necessary, and then such bodily waste can be flushed out of the device so it can be clean and reused.

Another object of this invention is to add comfort, convenience, cleanliness, improved hygiene and sanitation for those who can't use a toilet at the time of use, compared to traditional diapers, traditional bedpans, absorbent pads, urine bottles, and receptacles.

Yet, another object of the advanced diaper device is to greatly minimize or completely do away with what is known as diaper rash.

Another object is to fully aid/assist females in managing menstrual cycles, to provide menstrual management.

Another valuable object of this invention is to dramatically reduce the possibility of urinary tract infections (UTI), which are more likely to occur with traditional diapers.

### The Advantages

The advanced, high-tech diaper presented herein provides an easier, safer, more convenient, sanitary method for managing bodily waste for proper disposal. This is an invention idea that would better capture, collect, contain, and dispose of bodily excrements, reducing the chances of contamination beyond the advanced diaper system.

One of the greatest advantages of my invention and one of its winning features is that the enclosed waste capture chamber and urinal are flushable. That is, this advanced diaper device has a built-in flushing system, whereby water and other sanitizing fluids, enzymes, and deodorizers can be flushed throughout the system to adequately sterilize and clean it.

Another leading advantage the advanced high-tech diaper has over many other devices is it can be worn and used by both males and females and of any age. Very importantly, it may be worn and used by those who are disabled, handicapped, paralyzed, incapacitated, unconscious, debilitated, or mentally challenged. It may be used in any position, whether standing, seated, inclined, lying flat on one's back, front, or side. It may even be safely and effectively used while turned upside down or while in high, low, or zero gravity.

Another valuable advantage of my invention is that it serves to handle both urinary and fecal matter management, along with menstrual bleeding or vaginal discharges of other bodily fluids. Most bodily waste collection devices that are sold on the market or that have been patented only handle urine or fecal matter collection, but very rarely both and in my view never adequately.

This new advanced diaper system will greatly reduce the amount of direct contact the user's skin comes into with urine and fecal matter, thus helping to greatly reduce discomfort, skin irritation, traditional diaper rash, sores, urinary tract infections caused by bacterial contamination of the genitals—more likely to occur among those wearing traditional disposable diapers and non-disposable cloth diapers—and disease, and therefore making it far more hygienic.

Another important and valuable advantage this invention has over prior art is it would help dramatically reduce the waste of disposable diapers and absorbent pads that are harmfully filling up the landfills. My invention can be used repeatedly and could be used by additional siblings born into a family and even handed down from one generation to the next or used by new patients in a hospital as current bedpans are repeatedly used, yet much easier to clean.

Another advantage is that this advanced diaper invention can be used multiple times before needing to be emptied or cleaned, due to the sizable waste chamber built in. Different embodiments will have varying capacities. Some will be large, while others will be more streamlined and smaller in size so as to be more discreet and less noticeable, especially for females who wear one to manage their menstrual bleeding.

Another advantage is that this advanced diaper system is portable, equipped with its own rollable compactable, carriable cart, and therefore it could easily be taken anywhere the user goes or is taken by a caregiver. The water tank, waste collection container, and cart could all collectively be as small as a carry-on suitcase.

Another advantage of this invention is that it's a very closed system, thus reducing urine and fecal contamination and the transfer of pathogens. The advanced diaper device is designed to contain bodily waste better than previous devices and methods with the use of non-disposable materials and water resistant seals around the genitals so that it will prevent significant spills or leaking of bodily excrements, including unpleasant gases and odors. All bodily waste can move from the body to the advanced diaper device, to a storage container by means of a conduit, and then to a toilet or sewer pipe. It may also go directly from the advanced diaper device to the toilet if the user is able to sit on a toilet or be placed on a toilet. This becomes advantageous to those who can sit on a toilet, yet can't control their bowel movements or predict them. Newborn babies may need to be placed over a special, lightweight, safety seat cover insert that sits on top of the open toilet seat to prevent a small baby from accidentally falling into the toilet.

A valuable advantage compared to previous bodily waste management systems is this invention prevents most of the unpleasant odors from escaping the diaper, since it is a very closed system keeping all waste very well contained within a capture and storage chamber. However, it does have a bodily gas release flue and valve system, whereby bodily gases can be manually released at a more appropriate time and place once those gases build up to certain levels of pressure as well as a passive automatic gas release mechanism if the pressure ever reaches too high.

An important advantage and difference this invention has when compared to past inventions is that its inner and outer shell, along with its urinal and waste collection chamber, are comprised of rigid and/or semi-rigid materials, which prevents urine and stool and other bodily waste from being compressed and pushed back up through the apertures they entered.

Another great and valuable advantage is that this invention is designed to provide improved ease of use with little to no assistance for many users to operate. So in many cases, these advanced diaper units could be operated and cleaned by the user without assistance, which would help to preserve some degree of dignity, privacy, and control over one's own bathroom activities.

Yet another valuable advantage of my invention is that it will help to dramatically reduce the workload for nurses and caretakers when it comes to managing the bodily waste of those they care for as well as make it more tolerable for all involved.

The advanced diaper device also reduces the degree of contact caretakers come in with dangerous fecal-borne pathogens like Salmonella, E. coli, Giardia, hepatitis A, campylobacter, adenovirus, coxsackie virus, and Shigella (Shigellosis often occurs in toddlers who are not fully toilet-trained).

Another valuable advantage of this invention is that it's far less intrusive and thus more private. Most embodiments are also non-invasive unlike other inventions like catheters, urine wicks, and devices or methods involving tubes that are inserted into the urethra or rectum by nurses or caregivers.

The present invention could be extremely useful for those in remote places or during difficult tasks such as while mountain climbing, sky diving, hot air ballooning, or on long drives where restrooms are not available. One embodiment may even allow one to use this more sophisticated diaper system being discussed while swimming or deep sea diving as long as all the seals against the user's body were made well enough, possibly involving water resistant neoprene and silicone materials. At lower depths of water, the surrounding pressure would actually help to hold the diaper more tightly against the wearer's skin.

Another advantage is that this diaper device is designed to fit snuggly against the user's body. In some embodiments it uses silicone, neoprene, and vinyl covered foam cushions to provide comfort and adequate seals in all the right places.

Yet, another advantage of this invention is that it doesn't absolutely require electrical power from any source to operate. Some embodiments may incorporate electrical power from a wall outlet or a battery or both to power pumps, vacuums, and other electrical functions such as lights, indicators, circuits, and sensors.

Another notable advantage of this advanced diaper invention is that it greatly reduces the overall costs when considering the entire duration of use during toilet training or illness or conditional needs compared to having to buy disposable diapers for the same length of time.

Another great advantage this invention has over many others is it can more adequately help those who struggle and are routinely burdened with mild or severe diarrhea, such as those suffering from Crohn's disease, ulcerative colitis, celiac disease, inflammatory bowel syndrome, SIBO (small intestinal bacterial overgrowth), food poisoning, or any other gastrointestinal condition that cause unpredictable bowel movements, especially those that are very liquefied.

The water resistant seals of the present invention are advantageous, because they will be made of silicone-based elastomers with a tacky (slightly sticky) texture and moderately adhesive property without any applied surface glues or topical adhesives that could wear off or become permanently ineffective over a short term of use.

It's important to also point out that this advanced diaper device is to use applied force to these mildly sticky seals against the user's body due to a snug fit or by a user or caretaker holding it against the body of a user when using the device as a bedpan, so that adhesion is not the only force providing the seal between a user's skin and the device. Prior devices have and do use adhesive strips and patches that are too aggressive and can be painful to remove, especially for users with a lot of body hair, they are often toxic, don't provide an adequate seal by themselves due to the curved contours of the body and perspiration, and such adhesive strips and patches don't tend to last long and can't be reused over and over. Soft, flexible, cushion-like seals with a naturally mild sticky surface texture gently compressed against the body is needed, seals that are capable of easily conforming to the contours of the body (much like sitting on a thick sticky donut) is the solution.

I believe certain embodiments of my invention could help future astronauts and overall manned space flight efforts from all countries, including private commercial space flight programs, by solving many of the problems they have faced or will face, especially in terms of contamination and containment in low to zero gravity. My advanced diaper device could also be conveniently worn within a space suit during space walks or extravehicular activity.

These, along with additional advantages and objects of this current invention will become apparent, if not already, after reviewing the following detailed drawings and descriptions of the disclosed embodiments.

### BRIEF DESCRIPTION OF DRAWING FIGURES

Here are brief descriptions of the drawings and illustrations I've provided to help better communicate the concept and possible look of my invention and embodiments.

FIG. 1 is a perspective view of my invention being discussed with some shading, textures, and tones to help illustrate the overall look of the diaper device, which includes possible cushioned padding, an anus saddle bib, and a fly-away urinal crotch door to better see the inside.

FIG. 1D¹ is a perspective view of the initial embodiment of the advanced diaper device with its urinal faceplate and seal in a contracted position to show what it would most likely look like when it is clean and dry.

FIG. 1W¹ is a perspective view of the initial embodiment of the advanced diaper device with its urinal faceplate and seal in an expanded position to show what it would most likely look like when it is saturated and wet with urine.

FIG. 1D² is a perspective view of an alternate embodiment of the advanced diaper device with its urinal faceplate and seal in a contracted position to show what it would most likely look like when it is clean and dry.

FIG. 1W² is a perspective view of an alternate embodiment of the advanced diaper device with its urinal faceplate and seal in an expanded position to show what it would most likely look like when it is saturated and wet with urine.

FIG. 2 is a cross-sectional perspective view without the padded cushions and layered fabrics to better expose the inner parts and how they relate to one another, along with a back view of the urinal crotch door and a waste drain hose as it might engage the diaper device to be connected to the waste chamber drain.

FIG. 3 is another cross-sectional perspective view very similar to the one in FIG. 2, only without a urinal crotch door and waste drain hose, as well as portions of the inner diaper shell support framework and flushing tubes network that is fully revealed in FIG. 4.

FIG. 4 is a perspective view that shows one possible configuration of the inner diaper shell support frame and flushing tubes network.

FIG. 5 is a side view of the advanced high-tech diaper device to show it from another angle.

FIGS. 5a-5c are additional side views of the advanced diaper device, yet without the waste chamber seal liner, to show the waste chamber belly seal and alternate positions of the waste chamber belly as it moves to increase the volume of the waste chamber.

FIG. 6 shows a cross-sectional side view of the advanced diaper device with a waste drain hose fully attached and with the waste chamber drain valve in open drain mode and the flippers in the fully opened position.

FIG. 7 shows a straight on front view of the advanced diaper device as well as phantom lines to show an alternate position of the waste chamber belly.

FIGS. 7a-7c are front views of the advanced diaper device showing alternate positions of the waste chamber belly as it moves to increase the volume of the waste chamber for increased bodily waste.

FIG. 8 shows a bottom view of the advance diaper device that showcases a full bottom view of the waste chamber drain, its support lever, and its lid which is moved away in an off position, depicted with projection lines.

FIG. 9a shows a cross-sectional side view of one of many possible designs and/or embodiments for the waste chamber drain valve with its lid in an on position to show how they interlock together as well as an alternate off position of the lid, indicated with phantom lines.

FIG. 9b shows another cross-sectional side view of one of many possible embodiments for the waste chamber drain valve without its lid so as not to obstruct the view of its securing ridges.

FIG. 9c shows a tilted cross-sectional view indicated by section lines in FIG. 9d to help better show the curved shape of the valve and flippers.

FIG. 9d is a top view of one possible design and/or embodiment of the needed waste chamber drain valve with sectional lines to indicate the direction and sectional view shown in FIGS. 9a, 9b, and 9c.

FIG. 9e is a cross-sectional side view, indicated by section lines in FIG. 9k, of the waste chamber drain valve in a closed position to provide a reminder of what it looks like and as a comparison to the same drain valve in the open position in FIG. 9f

FIG. 9f is a cross-sectional side view, indicated by section lines in FIG. 9k, of the waste chamber valve in an opened position.

FIGS. 9g-9o are bottom views of varying possible designs and embodiments of a waste chamber drain valve showing their underside.

FIG. 10a-c shows partial cross-sectional side views of the advanced diaper being discussed and one possible embodiment of one of its possible counterparts, a waste drain hose, to illustrate how the two come together and interconnect, along with the opening of said waste chamber drain valve illustrated in FIGS. 9a-9o.

FIG. 10d is a cross-sectional side view of said waste chamber drain valve without its securing lever, sectional lines indicate the direction of view in FIG. 10e, as a comparison to FIG. 10e.

FIG. 10e is a top view of said waste chamber drain valve using hidden lines to show the relationship and placement of the securing ridges.

FIG. 10f is a side view of said waste drain hose.

FIG. 10g is a top view of said waste drain hose.

FIG. 10h is a side view of said waste drain hose interconnected with said waste chamber drain valve illustrated in FIG. 10d and FIG. 10e.

FIG. 10i shows said waste drain hose connected to said waste chamber drain valve and turned into the secure position using two sets of hidden lines; one set to depict the securing ridges of said waste drain hose in a turned position and a second set of hidden lines to depict the said waste drain hose nozzle ridges.

FIG. 11a is a perspective view of the anus saddle for the first embodiment for the advanced diaper device.

FIG. 11b is a cross-sectional view showing the back half of the anus saddle illustrated in FIG. 11a, indicated by section lines in FIG. 11d, to showcase its anatomy.

FIG. 11c is a cross-sectional view of said anus saddle to showcase its components.

FIG. 11d is a top view of said anus saddle to further show and communicate its shape and purpose.

FIGS. 11e-11g are perspective views of alternate possible embodiments of an anus saddle for the advanced diaper device, showing varying shapes.

FIG. 11h is a perspective view of an alternate anus saddle design that combines the concept of an anus saddle and a urinal crotch seal as a single molded part instead of two separate parts.

FIG. 11i is a perspective view of a possible anus saddle companion part referred to as an anus saddle seal aligner.

FIG. 12 shows the main components of the overall advanced diaper system, such as an advanced diaper device, a waste drain hose, a flushing hose, a water tank, waste collection container, and a portable cart stand to show how they all relate and work together.

FIG. 13 is a large, shaded, front view of a baby wearing the advanced high-tech diaper.

FIG. 14a is a perspective view of a two year old wearing the advanced high-tech diaper.

FIG. 14b is a front view of a two year old wearing the advanced high-tech diaper.

FIG. 15a-15d show shaded illustrations of a mother preparing to flush her child's advanced high-tech diaper.

### REFERENCE NUMBERS IN DRAWINGS

10—Advanced High-Tech Diaper
12—Hooks Strap
15—Interior Cushion Padding
15a—Back Lumbar Cushion
15b—Shell Cushion Liner Pad
15c—Air Ventilation Channel
15d—Central Seat Cushion
15e—Front Abdominal Cushion
16—Air Vent
18—Bodily Gas Indicator
18a—Bodily Gas Indicator Peg
18b—Secondary Bodily Gas Indicator Bulb
18c—Bodily Gas Indicator Tube
18d—Primary Gas Indicator Bulb
18e—Bodily Gas Indicator Support Mantel Hole
20—Gas Pressure Release Valve Device
20a—Gas Pressure Release Valve and Knob
20b—Gas Pressure Release Valve Flue Tube
20c—Gas Pressure Release Valve Filter
21—Expandable Waste Chamber Belly Seal Liner
22—Diaper Cradle Shell Casing
22i—Interior Diaper Shell Casing Surface
22e—Exterior Diaper Shell Casing Surface
23—Waste Collection Chamber Pot
24—LED Waste Chamber Light Switch
25—Waste Chamber Drain Valve
25a—Top Waste Chamber Drain Valve Securing Ring
25b—Bottom Waste Chamber Drain Valve Securing Ring
25c—Waste Chamber Drain Valve Securing Ridge
25d—Waste Chamber Drain Valve Support Shielding
25e—Waste Chamber Drain Valve Membrane
25de—Waste Chamber Drain Valve Flipper
25Mde—Master Chamber Drain Valve Flipper
25f—Waste Chamber Drain Valve Securing Lever Handle
25g—Waste Chamber Drain Valve Securing Lever
25h—Waste Chamber Drain Valve Lid
26L—Rear Left Expandable Viewport Seal
26R—Rear Right Expandable Viewport Seal
27L—Rear Left Viewport
27R—Rear Right Viewport
28—Expandable Chamber Belly Seal
29—Sensitive One-Way Check Valve
30—Anus Saddle Seal Valve
30a—Anus Saddle One-Way Bidet Valve Flap
30b—Anus Saddle One-Way Air-Flow Valve Flap
30c—Anus Saddle Bidet Channel
30d—Anus Saddle Air-Flow Channel
30e—Anus Saddle Valve Flap
30f—Anus Saddle Valve Fissure
30g—Anus Saddle Securing Flange
30h—Anus Saddle Bib
31L—Front Left Expandable Viewport Seal
31R—Front Right Expandable Viewport Seal
32L—Front Left Viewport
32R—Front Right Viewport
33—Bidet Flow Control Valve
34—Cushioned Leak-Guard Thigh Cuff
35—Bodily Waste Chamber Belly
35u—Urinal Drain Port
36—Loops Patch
38—Urinal Sink
39—Urinal Spill-Guard Ridge
40—Urinal Crotch Door
40a—Urinal Crotch Door Handle
40b—Expandable Urinal Faceplate Seal
40c—Company Brand Logo Placement Designation
40d—Urinal Crotch Door Faceplate
40e—Urinal Crotch Door Securing Snaps
40f—Super Absorbent Urinal Sponge
40g—Urinal Sponge Guard Cage
40h—Urinal Crotch Door Insert
40i—Urinal Sponge Guard Cage Fin Hinge
41—Urinal Crotch Door Notches
42—Urinal Crotch Seal
44—Urinal Crotch Door Gasket Seal
45—Urinal
46—Speaker Holes
48y—Mini LED Urine Indicator Light Bulb-Yellow
48r—Mini LED Urine Indicator Light Bulb-Red
48o—Mini LED Urine Indicator Light Bulb-Orange
50—Navel Flushing Hose Port
50a—Flushing Hose Port Securing Ridge
50b—Flushing Hose Port Gasket Seal
50c—Flushing Hose Port Inlet
52—One-Way Liquid-Flow Check Valve
52m—Mini One-Way Liquid-Flow Check Valve
54—Diaper Seat and Waste Chamber Cover Shield
540—Stool Passage Port Opening
55—Bidet
56—One-Way Air-Flow Check Valve
56m—Mini One-Way Air-Flow Check Valve
58—Front Diaper Shell Support Frame Stud
60—Waste Drain Hose
60a—Waste Drain Hose Nozzle
60a1—Waste Drain Hose Nozzle Securing Ridge
60a2—Waste Drain Hose Nozzle Finger Turn Grip Collar
60b—Waste Drain Hose Tube
60c—Waste Drain Hose Sleeve
61—Front Bottom Support Mantel
62—Rear Bottom Support Mantel
63—Front Top Support Mantel
64—Rear Top Support Mantel
65—Interior Diaper Shell Support Frame
66—Top Support Mantel Vent
68—Rear Diaper Shell Support Frame Stud
69—Flushing Tube
69m—Mini Tube
70—Flushing Tubes Network
71—Diaper Shell Support Frame Elbow Joint
72—Elbow Connector
72m—Mini Elbow Connector
73—T-Connector
75—Air-Drying Device and System
80—Portable Flushing Cart Stand
82—Flushing Water Tank
84—Bodily Waste Collection and Storage Container
85—Flushing Hose
85v—Flushing Hose Flow Control Valve
85L—Flushing Hose Flow Control Valve Lever
85n—Flushing Hose Nozzle
85r—Flushing Hose Nozzle Securing Ridge
85i—Flushing Hose Nozzle Insert

### DETAILED DESCRIPTION OF THE INVENTION

The following is a more detailed description of the embodiment usually discussed herein, along with other possible embodiments, but the full scope of this invention should not be limited by these embodiments discussed. They are only meant to serve as possible examples.

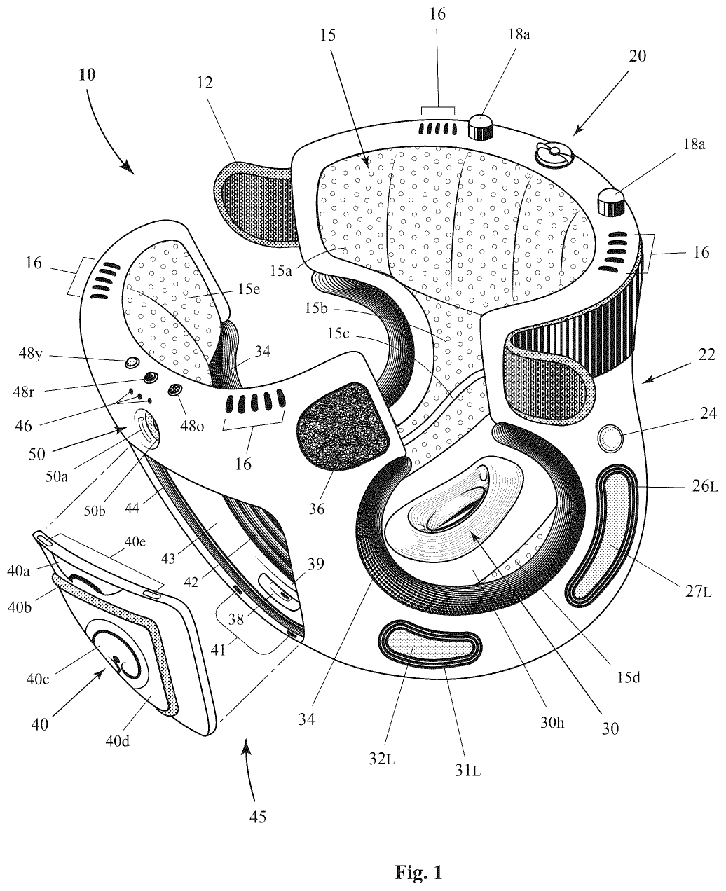

As illustrated in the figure drawings, one possible embodiment of my invention of the advanced hi-tech diaper system, such as shown in FIGS. 1-3, and 6, comprises a double-walled plastic shell casing 22 (sometimes referred to as a diaper cradle or cradle shell). Therefore, the diaper cradle 22 has an inner interior shell 22i and an external exterior shell 22e that are curved and bent towards one another and joined at their seams by means of heat sealing or glue to serve as the main body of the device. Each shell surface is approximately 1 to 3 mm thick and made of a somewhat flexible polymer, such as a thermoplastic like polyethylene, and is either thermally shaped and molded into a desirable form using thermoforming techniques or is made by means of pour or injection molding and possibly with thermosetting plastics.

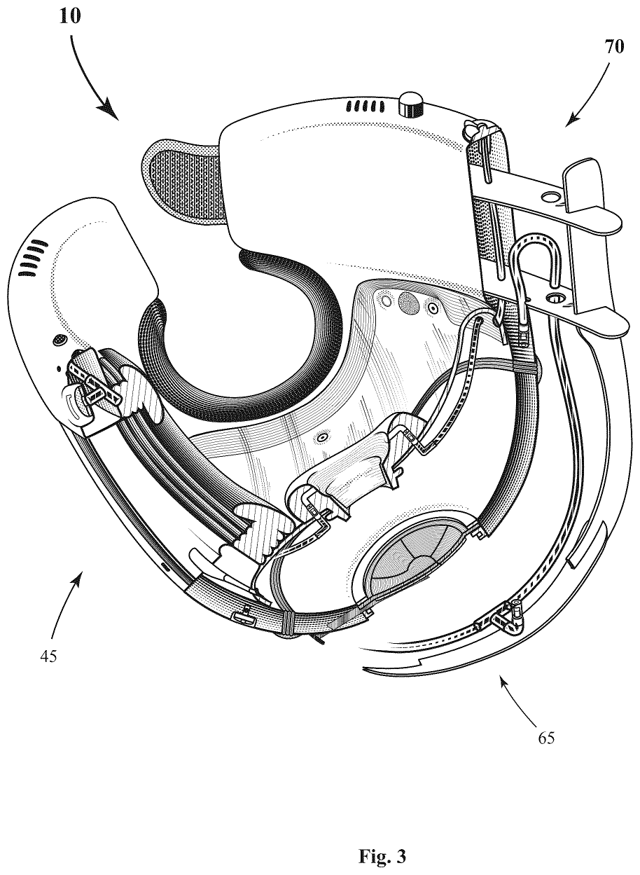

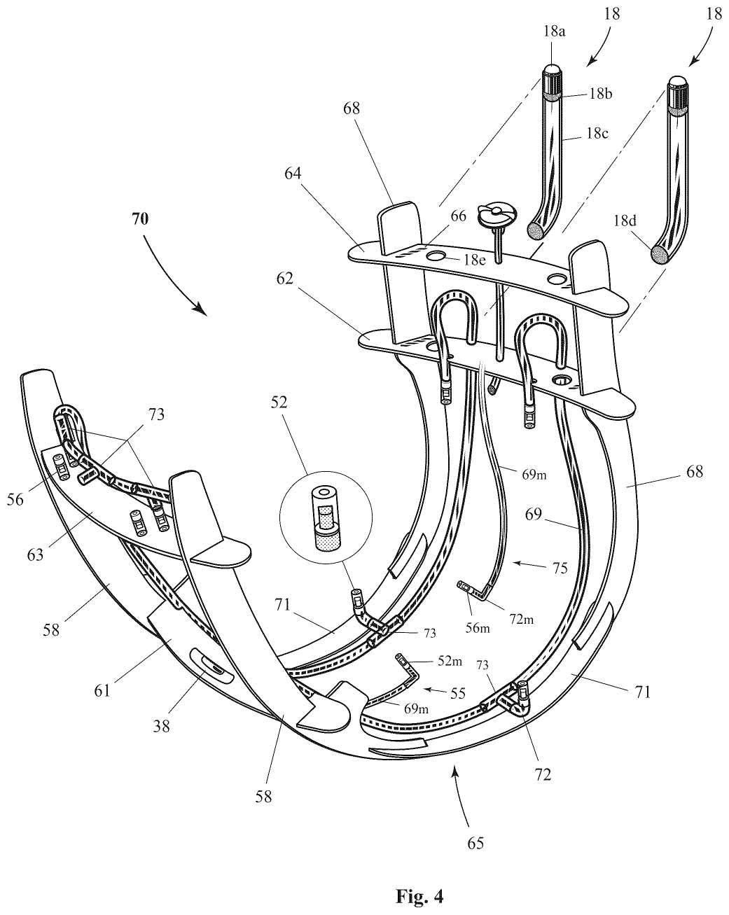

Inside the double-walled shell 22 is a cavity or available space, which may vary in thickness from one embodiment to another and from one location of the diaper shell 22 to another in any particular embodiment (anywhere from 0.5 cm to 3 cm wide). Inside this available space between the interior diaper shell 22i and exterior diaper shell 22e is a flushing tubes network 70 and a plastic support frame 65 as shown in FIGS. 3 and 4. In other words, the shell 22 is placed over and around a flushing tubes network 70 that is attached to a plastic support frame 65, thus, making the overall diaper device 10 a relatively rigid structure with some flexibility.

The flushing tubes network 70 involves acrylic tubes 69, 69m, plastic connectors 72, 72m, 73, 73m (elbow or L-shaped and T-shaped), and one-way check valves 52, 52m, 56, 56m. It provides a bidet 55 and air-drying system 75 that could be electrically heated. The diaper shell 22 support frame 65 is made from about ten plastic profiles cut from plastic sheets about 2 mm thick, give or take, and then assembled. Each profile cutout shape has a notch or groove that can interlock with a corresponding piece and then possibly be glued if necessary. There are two rear diaper shell support frame studs 68 and two front diaper shell support studs 58. These four studs interlock into two lower central diaper shell support frame elbow joints 71 that bend in the perpendicular direction to the studs' direction of bend to allow the overall frame 65 and diaper shell 22 to bend and flex a little in a closing and opening manner around the wearer of the diaper device 10. The rear studs 68 are interconnected with support mantels as well, a rear top support mantel 64 and a rear bottom support mantel 62. The front studs are also interconnected with support mantels, a front top support mantel 63 and a front bottom support mantel 61. This framework 65 could also be created by means of injection mold techniques or other mold techniques. But I'm hoping to make most if not all the plastic parts of this diaper device and system with thermoforming thermoplastics, thus reheatable, reshapable and recyclable as much as possible.

The support frame 65 is designed to support the body of the diaper shell cradle 22 and to help hold its shape as well as provide support mantels or shelves for installing needed parts like flushing tubes 69, 69m, urinal sink and drain 38, bidet tube 69m, air-drying conduit 69m, one-way check valves 52, 52m, 56, 56m, bodily gas indicator tubes 18c, bodily gas release valve 20, bodily gas release flue 20b, and top support mantel air vents 66, etc. These parts and their arrangements will be discussed further and in more detail after certain other components have first been described in order to better understand their relationship. The support mantels may also help support and secure water storage bottles/tanks, electric heating coils, electric motorized fan, and battery storage packs for other embodiments of this diaper device, which are not shown in any of the drawings.

This diaper shell 22 is to be shaped so that it curves around the thighs of a user and should be comfortably worn for many hours if necessary. These curved areas of the diaper shell 22 for a user's thighs are to be lined with cushioned leak guard cuffs 34. Each cushioned leak guard cuff 34 should be soft and flexible and made of water resistant material, such as a synthetic foam polymer or rubber and may include vinyl coverings. They are to be attached to the diaper shell 22 by means of an adequate glue bond and/or fasteners like staples or rivets or sewn stitching. These cuffs 34 are meant to prevent any rare, yet possible accidental leaks of bodily waste.

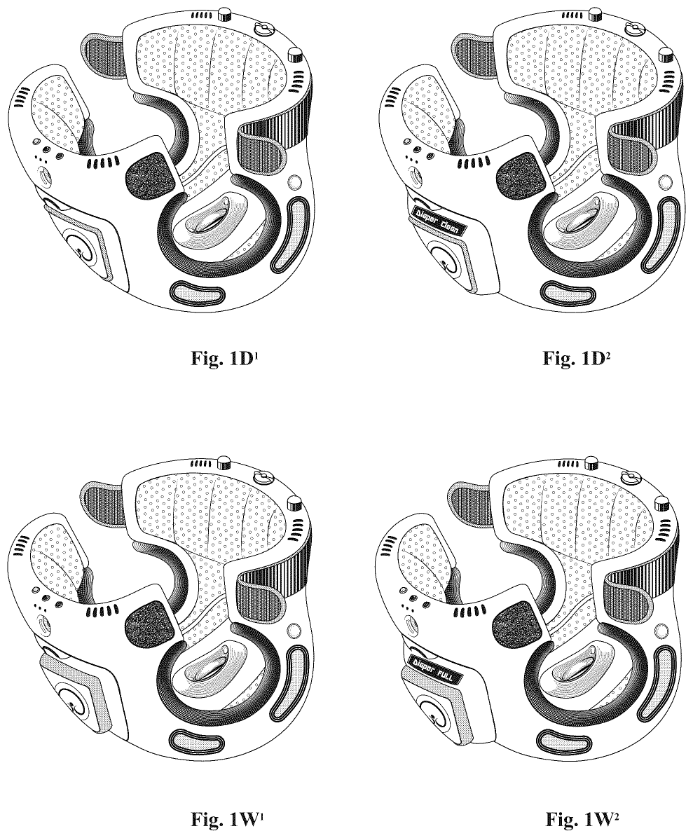

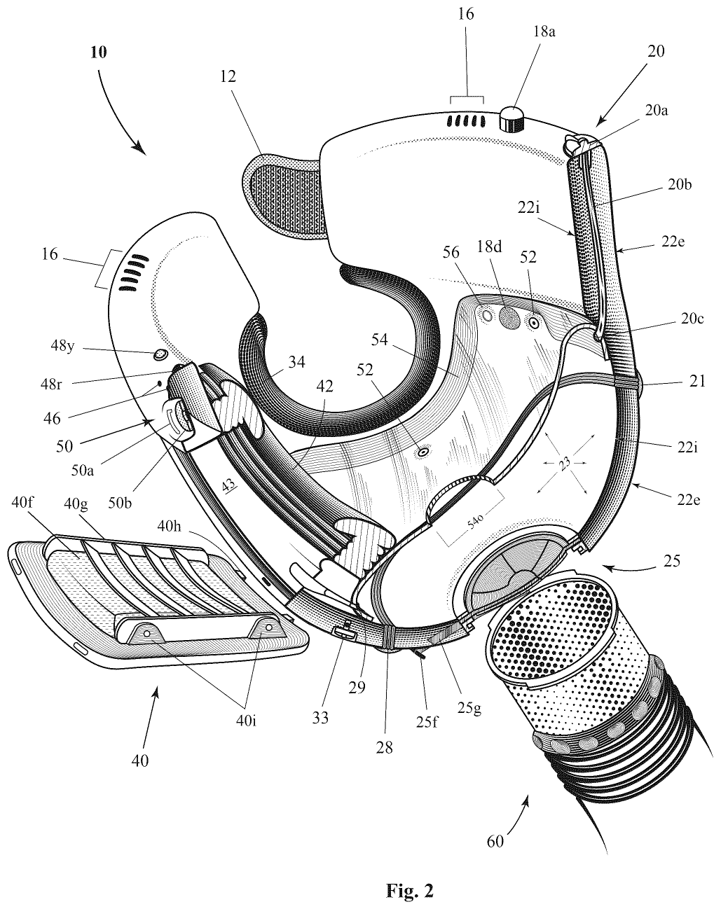

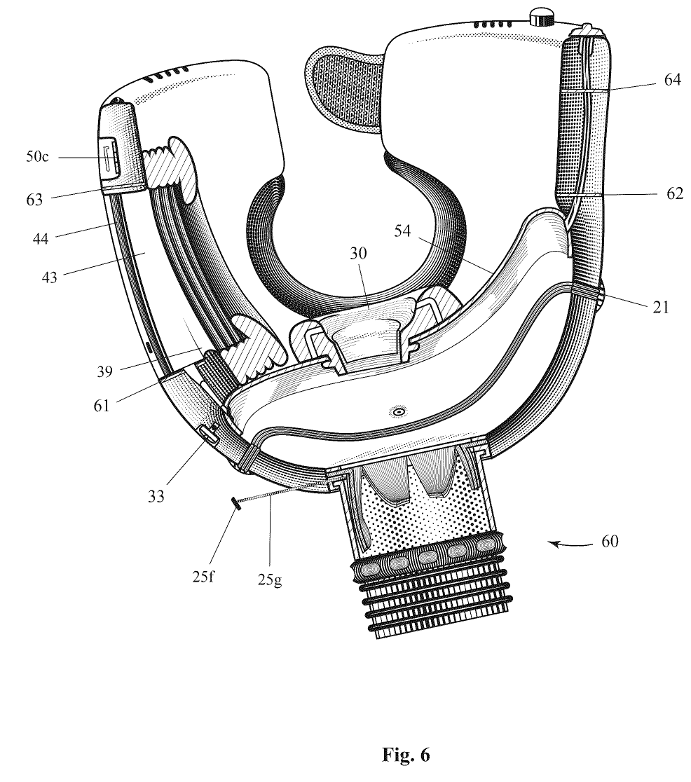

The next important feature is the diaper seat 54 as shown in FIGS. 2 and 6, which is a rigid curved plastic membrane, ideally transparent or semi-transparent or translucent to allow for visual inspections when necessary. This diaper seat 54 has a folded edge all around to be either glued or heat sealed to the interior diaper shell 22i of the diaper cradle shell 22 to form a waste collection chamber pot 23, a container for collecting bodily waste, such as urine, menstrual blood, vaginal discharge, semen, and stool. Thus, the diaper seat 54 may also be referred to as the waste chamber cover shield 54 or simply chamber cover 54 or chamber top 54. The diaper seat 54 has an adequate hole referred to as a stool passage port opening 540 about 4 cm to 7 cm in diameter for stool to pass through and into the waste collection chamber pot 23.

The waste chamber 23 has a waste drain valve 25 as shown in FIGS. 2, 6, 8 and FIGS. 9a-9o and FIGS. 10a-10i. It is about 5 cm to 6 cm in diameter. It's comprised of a top capping ring 25a and a bottom securing ring 25b which has securing ridges 25c that help to hold in place a drain hose 60 when one is attached. Sandwiched in between the waste chamber drain capping ring 25a and securing ring 25c is the waste chamber drain valve membrane 25e that is flexible and made of a synthetic material like rubber. This valve membrane 25e has support shielding 25d made of thin plastic attached to it by means of gluing or by means of interconnected molding. Both the membrane 25e and shielding 25d are cut and divided into sections called flippers or petals 25de, anywhere from 2 to 8 divisions or even more if that proves to be better in some way, so that they may open and close like the petals of a blossoming flower when the diaper device 10 needs to be flushed and cleaned.

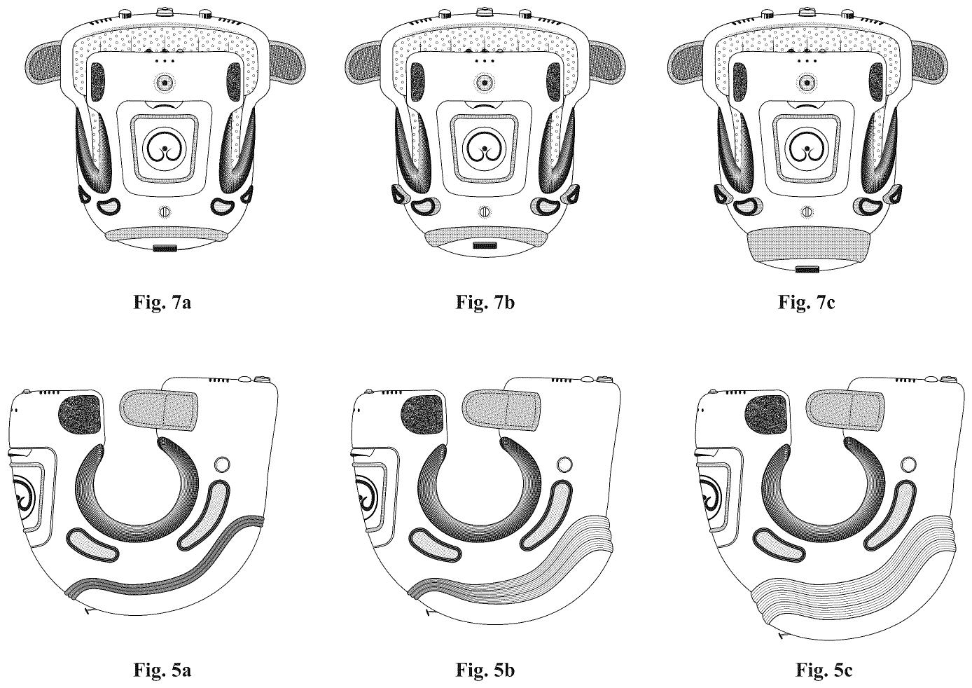

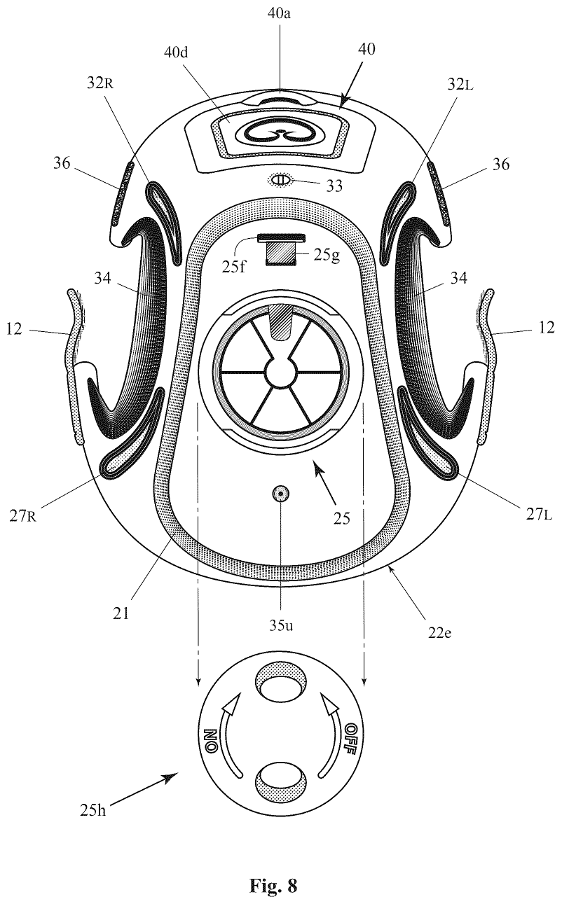

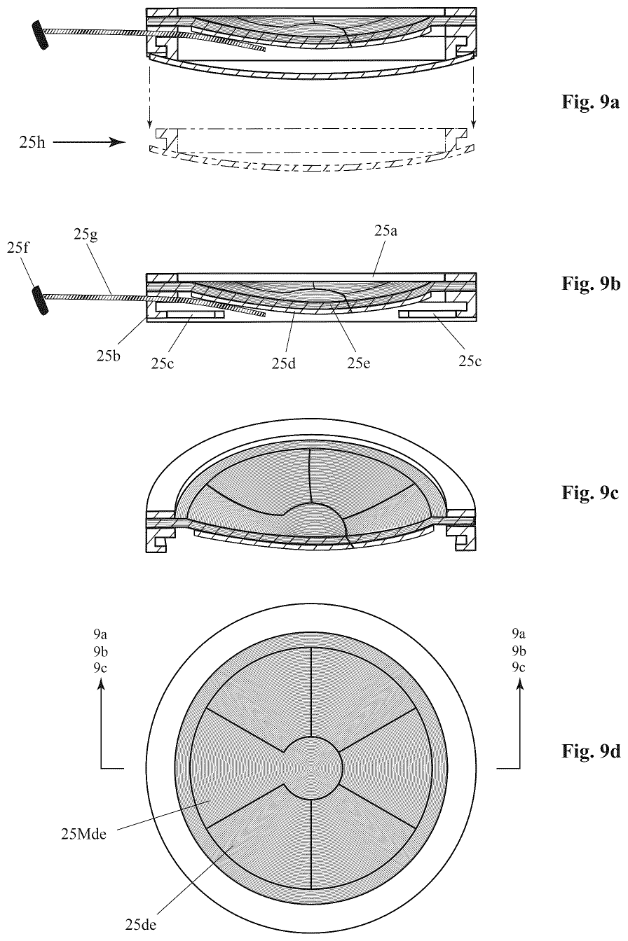

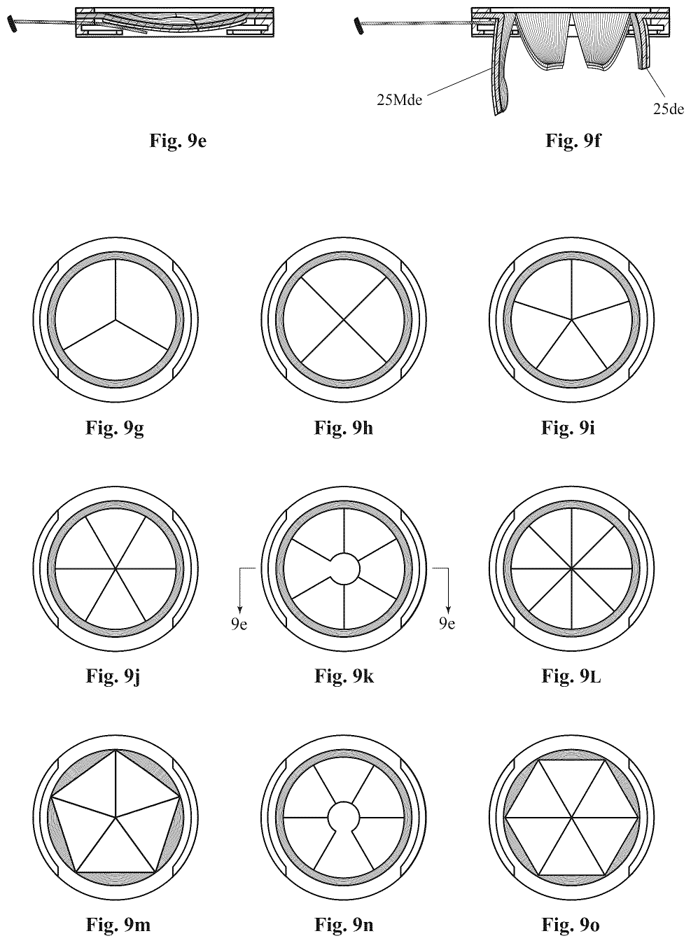

FIGS. 9g-9o show different possible divisions and designs of a waste drain valve 25; one in particular being the one shown in FIG. 9k with a master flipper or master petal 25Mde, which may also be referred to as a parent petal 25Mde, because it is the one I'm currently discussing and using as an example in this first possible embodiment. The purpose of having a master flipper 25Mde is to help insure proper closure of all the flippers 25de, while at the same time providing a better seal among all the flipper seams and a good central pressure point for the waste drain securing blade 25g. The master flipper 25Mde will be larger and thus heavier and take longer to close compared to the smaller lighter flippers 25de that need to close first. In some embodiments, the master flipper or master petal 25Mde may have guiding appendages at its lower base to help corral any smaller flippers 25de that get stuck or delayed in closing. The smaller flippers 25de that neighbor the master flipper 25Mde may also have appendages of their own (on the far side) to help corral their neighboring smaller flippers 25de. These appendages are not illustrated anywhere in any of the figure drawings. The waste drain securing blade 25g has a small handle 25f that can be used to pull it out or into a secure position. This waste drain valve 25 also has a plastic lid 25h that is to be screwed into place so as to provide added security in case of any rare accidental leaks.

The adequately sized hole or stool passage port opening 540 in the diaper seat 54 should allow a soft, flexible, rubber anus saddle seal valve 30 (often referred to as simply an anus saddle 30) to be inserted and secured and later removed if necessary to be cleaned, replaced, or to gain access to the waste chamber 23. The anus saddle 30 is made of a silicone-based elastomer to give it a sticky, tacky, texture and a natural adhesive property when clean so as not to use added glue substances that can wear away, similar to the elastomer toys popularized in the 1980's called Wall Crawlers and Wall Walkers. More recent products like Restickable Dots, used to mount pictures, use the same type of technology. They too are made of rubber-based adhesive substances that are non-toxic, acid free, and odorless but have a very sticky texture while clean and dry.

As illustrated in FIGS. 1, 6, and FIGS. 11a-11i, the anus saddle 30 has a narrow fissure 30f made primarily by two thin valve flaps 30e that come together and fold downward to serve as a one-way valve to allow fecal excrement to easily pass through but not easily back the way it came. In this embodiment, the anus saddle 30 also has two channels that run through it and one-way valve flaps 30a and 30b; one air-flow channel 30d and air-flow valve flap 30b is to serve as an air-flow and air-drying system that takes in external air during and after a flush so as to allow proper air flow so the waste chamber 23 can be more easily emptied and to help dry off the anus area of the diaper user. In some embodiments, the inflow of air may be heated by electric coils to a safe degree for added comfort and pleasure. The second is a bidet channel 30c with a bidet valve flap 30a, which allows for a flow and/or spray of water to act as a bidet to help clean the anus during and after a flushing.

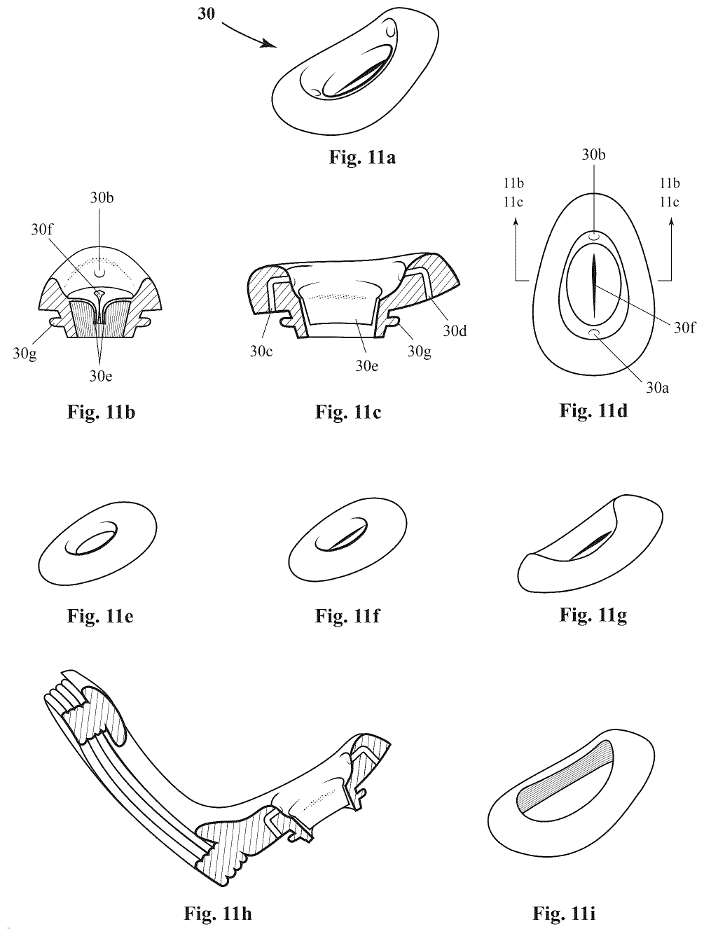

The anus saddle 30 can be secured in place into the stool passage port hole of the diaper seat by means of its lower flange 30g. This flange 30g is part of the anus saddle 30 likely as a single molded part. The anus saddle 30 may be fashioned in a variety of shapes and sizes and materials, but for now let me say it could be made of a synthetic rubber like silicone, which when clean can be made to have a natural, non-toxic, sticky adhesive quality and is meant to be pressed up against the skin and body of the user creating an adequate seal to prevent leakage of bodily waste. A soft wax or purified petroleum jelly, such as Vaseline® could be applied to the anus saddle 30 to improve the seal between it and the user's anus if necessary. A few more possible embodiments of an anus saddle of varying shapes can be viewed by reviewing FIGS. 11e-11h and an anus saddle seal also made of silicone can be viewed in FIG. 11i. The anus saddle seal would be applied around a user's anus before having the advanced diaper 10 put on to form an even better seal, but this additional seal may not be necessary. There are of course other possible shapes and sizes for an anus saddle 30 but the examples provided can give a close approximate idea of their shape and purpose but should not be limited by those examples.

The anus saddle 30 should be made large enough and flexible enough so that if the diaper user moves around the seal will not be significantly broken. If some leakage ever occurs, it could be captured and absorbed by an anus saddle bib 30h that can optionally be placed just under the rim of the anus saddle 30, yet above the diaper seat 54, also referred to as a waste chamber cover shield 54. This bib 30h could be made of a combination of absorbent materials and water resistant materials. It may be made to be washable and reusable, such as cloth made of cotton or wool or made to be disposable using cotton, wool, and/or synthetic materials. In this embodiment, a thin top layer of water resistant netting with layers of water absorbent materials after, and then with a bottom water resistant material.

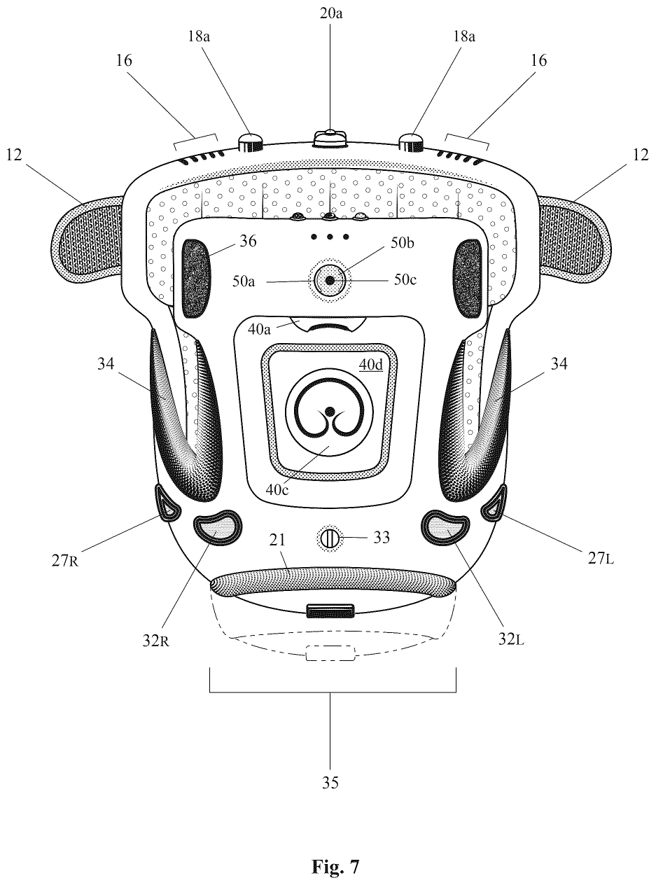

Now that certain parts of the diaper have been discussed in some detail, I'd like to return to discussing the parts of the flushing tubes network 70, viewable in FIG. 4, and how they are connected or installed into the diaper shell support frame 65. Two air-flow one-way check valves 56 should be inserted into the top forward support mantel 63 to allow air to be drawn into the urinal 45 space as well as into the bodily waste collection chamber 23, especially during a flushing. When it comes to fluid dynamics (air is considered a fluid) and simple physical laws, in order to empty one container of any matter, whether a sold, liquid or gas, that void must be replaced with new matter or the contents will try to remain in place. At the rear of the diaper device 10, two more airflow one-way check valves 56 are inserted through the interior shell surface 22i and into the waste chamber 23. It can draw air thanks to the air ventilation vents 66 cut into the back support mantels 62, 64. As part of the flushing tubes network 70 inside the diaper shell 22, about 6 one-way liquid-flow check valves 52 should be installed. These valves would likely be inserted into the proper ends of acrylic flushing tubes 69 and possibly glued in place. Some of these valves may use plastic connectors as well, such as those that are L-shaped 72, 72m or T-shaped 73 to help change their direction from the tube they've been inserted into. There should be two one-way liquid-flow check valves 52 inserted into the forward top support mantel 63, two at the bottom of the support frame 65, where the support frame elbow joints 71 are, and two that are attached to the rear bottom support mantel 62 and which are inserted through the interior shell surface 22i to protrude into the waste chamber pot 23. In addition to the flushing tubes 69 and their one-way check valves, there are two smaller acrylic tubes 69m with mini one-way check valves. One is a mini one-way liquid-flow check valve 52m up front with a plastic elbow connector 72m used for the bidet service, which includes a flow control valve 33 (visible in FIGS. 2, 6-8). The other is a mini one-way air-flow check valve 56m with a plastic elbow connector 72m to serve as an air-dryer 75. Both of these mini valves could be installed at the bottom of the waste chamber 23, but in the first embodiment discussed, they would be installed into the available channels 30c and 30d molded into the anus saddle 30.

The double-walled diaper shell 22 previously discussed, regarding this particular embodiment, will have multiple cutouts for view ports, expansion seals, flushing port, waste drain valves, gas release valve, indicator lights, indicator tubes, vents, LED light switch and bulbs, and a urinary crotch door, etc., which will all be discussed in some detail below.

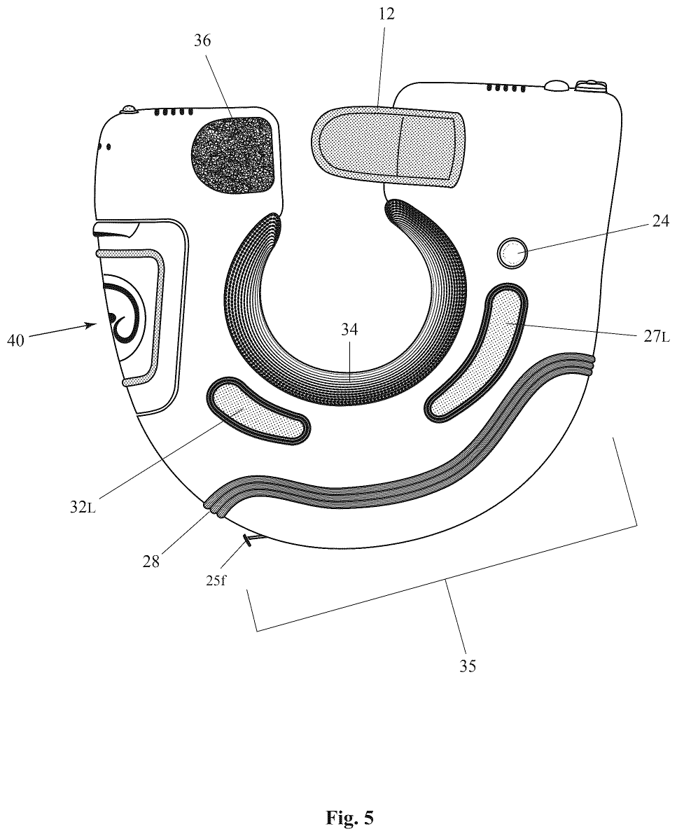

If you view FIGS. 1, 5, 5a-5c, 7, 7a-7c and 8, you will see on both sides of the diaper cradle shell 22, in the front and in the rear, there are viewports 27L, 27R, 32L, 32R, which will allow for visual inspections into the waste chamber. These viewports are to be made of transparent acrylic plastic about a millimeter thick or less and attached to the diaper shell 22 with flexible, expandable rubber seals 26L, 26R, 31L, 31R, allowing the viewports to move outward to increase the volume of the waste chamber 23 as it fills up with bodily waste. These expandable viewports also serve to indicate that the diaper is likely full and needs to be cleaned. The viewport seals 26L, 26R, 31L, 31R should be attached with some form of adhesive glue and/or waterproof sealant that provides a strong enough bond and perhaps by means of fasteners like staples or rivets or sewn stitching. An LED light switch and/or button 24 can be installed within the diaper shell 22, along with an interior LED light bulb (not shown in any drawings) to provide interior lighting within the waste chamber so that caretakers may better inspect inside the waste chamber 23 to see if the diaper is full or needs to be flushed and cleaned.

In this embodiment, there is a waste chamber belly 35 that can drop down to expand the volume of the waste chamber 23. It is made similarly to the diaper shell 22 in that it is comprised of an inner surface or membrane and an outer surface, about 1 to 3 mm thick, joined together by glue or heat seal at their edges, again forming a cavity within of similar dimensions in terms of its width. The waste chamber drain valve 25 is located near its center. The waste chamber belly 35 is attached to the diaper device 10 by means of a rubber seal 28. This seal 28 may be corrugated or folded accordion style or may be a thin membrane folded in multiple places or simply very flexible, expandable rubber. The outer larger circumference of the seal would be attached to the bottom diaper shell opening that would be made for the waste chamber belly 35 and its inner smaller circumference attached to the waste chamber belly 35 itself in a way that prevents any leakage of bodily waste or flushing fluids. A flexible, stretchy, elastic waste chamber seal liner 21 made of synthetic woven fabric, such as spandex, may be fashioned around the rubber waste chamber seal 28 as an added barrier and to serve as another indicator that the diaper is full by using multi-layered, multi-colored fabrics. When the waste chamber belly 35 drops too low, indicating it is clearly full and needs to be cleaned, a certain colored fabric could be revealed and become visible.

Another important feature built into this diaper device 10 is a urinal 45, an open cavity or chamber equipped with super absorbent sponges 40f, a sponge guard cage 40g, a urinal sink 38, and one-way check-valves 52, 56, 29 for a user to urinate into. There is a curved urinal crotch door 40 made of molded plastic. The urinal crotch door 40 has a finger grab handle 40a and a faceplate 40d where an optional company logo 40c could be placed, which is attached to an expandable seal 40b made of a synthetic material like rubber that is attached to the urinal door 40. On the opposite side, facing inward when attached, is a urinal sponge guard cage 40g that holds a highly absorbent material like a super absorbent sponge 40f. Absorbent gel materials (AGM or hydrogels) or sodium polyacrylate powders, crystals, or beads could be used. However, they can be somewhat environmentally hazardous and would prefer to avoid them if possible. This sponge 40f starts out relatively thin when dry, but when it gets wet and saturated it expands greatly, pushing out the faceplate 40d and expandable seal 40b.

One can review FIGS. 1D¹, 1W¹, 1D², and 1W² to get an idea of how the faceplate 40d, seal 40b, and sponge 40f push out and expand. FIGS. 1D¹ and 1W¹ show the diaper of this first embodiment being primarily discussed at the moment, but FIGS. 1D² and 1W² show a more advanced embodiment with a larger urinal volume and a digital display panel. Other embodiments may not have a sponge 40f and sponge cage 40g at all. A user's urine would simply collect within the urinal 45 and then drain down the urinal sink 38. In the case of no sponge, a silicone insert could be used to help cushion the male genitals. Another possibility, regardless of whether a sponge is used or not, is using a water proof synthetic net or webbing to somewhat hold back the male genitals so as to prevent them from coming into contact and resting in any urine that has collected and pooled before having time to drain down the urinal sink 38 and into a collection container like the waste chamber 23 or in other embodiments a separate urine collection container (not shown in any of the drawings).

This crotch door 40 may be an independent part that can be easily and fully removed from the diaper device 10 and easily put back in place by a user or caretaker by means of physical notches 41, inserts 40h, and raised ridges called urinal crotch door securing snaps 40e that allow it to snap back into place or by magnetic means such as magnetic buttons or strips. The urinal crotch door 40 may also be hinged in a variety of ways and may swing open to the left, right, up or down, depending on what turns out to work best and be most advantageous. In some embodiments, there could be a door within a door, so that the main urinal door could be fully removed but on occasion a smaller hinged door could be opened and closed or vice versa. A larger urinal crotch door could be hinged but would have a smaller fully removable door built in. The idea of making the urinal crotch door 40 removable and/or able to be opened is so a user may freely use the bathroom in a more traditional way if and when possible. And it would allow the user or a caretaker to clean the urinal 45 area and the user's genitals if and when necessary as well as remove, replace, or clean the super absorbent urinal sponge 40f that is held within a urinal sponge guard cage 40g. The urinal sponge cage 40g could be secured by two sets of hinges 40i. Plastic pegs from the urinal sponge guard cage 40g would snap into corresponding holes in plastic hinge fins 40i on the main crotch door 40 to create these simplified hinges 40i. This way, a user or caretaker could simply squeeze the sides of the somewhat flexible plastic sponge guard cage 40g (near the top above the first set of hinges 40i) to remove the plastic pegs from the top plastic hinge fins 40i, for instance, to open the sponge guard cage 40g to more easily remove the urinal sponge 40f for cleaning or replacement.

One of the largest cutouts within the diaper shell 22 is for the urinal crotch door frame 43 likely a molded plastic insert that is glued in place (not adequately illustrated in the drawings). In the lower part of this door frame 43 sits a urinal sink 38 as well as a urinal spill-guard ridge 39 to help prevent urine from flowing back until it drains down the urinal sink 38 or is absorbed by the urinal sponge 40f. The urinal sink 38 is connected to a relatively small tube or conduit that leads to a very sensitive one-way check valve 29 that should allow urine and flushing fluids to enter into the waste chamber 23 but not back out, thus preventing any significant backflow. At the very outer lower edge of the urinal door frame 43 are two small notches 41 that should allow two small plastic inserts 40h on the bottom of the urinal crotch door 40 to fit in place during its attachment. One elongated central notch and insert could work as well. At the very outer upper top of the urinal door frame 43 are two recessed notches (not shown in any of the figure drawings) to allow the raised securing snaps 40e on top of the urinal door 40 to snap in place. On the outer surface area of the urinal door frame 43, located between the notches 41 and the urinal sink 38 should be a rubber urinal crotch door gasket seal 44 in the range of 0.5 to 5 millimeters thick to help provide a good leak proof seal when the urinal door 40 is attached in a closed position.

Further back inside the diaper device 10 and attached to the urinal crotch door frame 43 is a flexible rubber urinal crotch seal 42 (best viewed in FIGS. 2, 3, 6) that is to be pressed up against the user's body. When clean and free of dust, it should have a natural sticky adhesive quality and be able to effectively create a quality seal against the user's skin around the crotch and genitals. In this embodiment or perhaps others, a thin rubber membrane may be part of this molded rubber crotch seal 42 to act as an extra barrier and liquid-proof divider between the urinal 45 (that at times will be filled with urine) and the wearer's skin. This thin rubber membrane about 1 to 2 mm thick (not illustrated anywhere in the figure drawings) would have a hole just large enough to allow the genitals, male or female of proper age that corresponds with the diaper size, to show through. This would really be advantageous for when a user needs to urinate while lying on his or her back, since urine would spill back onto the user's unprotected groin area otherwise. A thin rubber membrane that essentially separates the user's skin from the entire urinal area, other than the adequately sized hole for the user's genitals, would certainly reduce the chances of experiencing urine backflow and getting wet. This crotch seal 42 should be thick enough and flexible enough so that if the user moves around, the seal would adequately be maintained well enough. It should ideally be soft and springy, possibly involving accordion type folds or corrugated, ribbed, walls that allow it to easily be compressed and then spring back into its original position.

Just below the urinal 45 is a relatively small plastic knob to the previously discussed flow control valve 33 to the bidet 55 mini tube 69m made of acrylic.

Moving above the urinal doorway, is the navel flushing hose port 50, roughly 1 to 2 cm in diameter and about 1 to 2 cm in depth (if not including a gasket seal). Inside is a rubber flushing tube port gasket seal 50b also about 1 to 2 cm in diameter and about 2 mm thick. This gasket seal has a hole which leads to a flushing hose inlet 50c that is connected to the flushing tubes network 70 inside the diaper shell 22. Along the sides of this flushing hose port 50 are plastic securing ridges 50a intended to help secure a flushing hose 85 in place during flushings.

Higher up above the navel flushing hose port 50 are three small holes (there could be more or less or a mere slit with fabric covering) to serve as speaker holes 46 or speaker outlet for any embodiment of this diaper device that includes an interior speaker to produce audible sounds to notify and to communicate to users and/or caretakers the condition of the diaper like whether the diaper is full or ¾ths full or half full or one quarter full, etc.

On the top front rim of the diaper device 10 may be one to three LED light bulbs or more to also communicate the condition of the diaper, such as whether there is urine saturation, gas buildup, or fecal matter buildup. For example, in this first embodiment, I use the example of having a yellow indicator bulb 48y on the right side of the diaper (left side to a caretaker facing the front of the diaper) to alert a user and/or caretaker that the super absorbent sponge 40f as well as the urinal sink 38 have filled with urine. A middle red indicator LED bulb 48r to indicate the presence of stool. And an orange LED indicator bulb 48o to indicate a buildup of gas that may need to be released using a gas release valve 20 located at the back top rim of the diaper. Of course the color of these bulbs and what information they communicate or bodily waste they correspond to can be altered in a variety of ways. In the center, on the top of the back rim of the shell cradle 22, there is a gas release valve knob 20a (indicated in FIG. 2) that can open and close a gas release valve device 20, which is connected to a small flue tube or conduit 20b to release built up bodily gas. This flue tube 20b can be made from a variety of materials, such as plastic or metal, and at its lower bottom end that reaches into the waste chamber there is a small gas pressure release valve filter 20c that allows only gases to get through and escape.

Adjacent to the gas release valve knob 20a are two bodily gas pressure indicators 18. They both consist of long plastic tubes 18c (likely acrylic tubes about 0.5 to 1 cm in diameter) installed into the rear support mantles 62, 64 and which bend into the waste chamber pot 23 towards their lower ends. At these ends that reach into the waste chamber pot 23 are gas indicator bulbs 18d, which are small rubber dome-shaped diaphragms. In other embodiments, they may be much larger, as wide as large coins, say 2 to 3 cm in diameter. At the top end of these plastic tubes 18c sits plastic pegs 18a with red color bands around their cylindrical form or any other color that can be used to indicate the diaper is full of gas and/or fecal matter. The indicating color can either be painted on directly or painted onto another material that is then wrapped around the pegs 18a and glued on. Just below these pegs 18a are a second set of rubber bulbs 18b adhered to the tube so as to create an air tight seal within. These gas pressure indicators 18 should be fashioned so that the pegs 18a can freely move up when gas pressure builds up within the waste chamber 23 and presses against the rubber gas indicator bulbs 18d, compressing the air inside the gas indicator tubes 18c and then onto the secondary gas indicator bulbs 18b. These pegs 18a should also be free to fall back into place once the gas has been released by the bodily gas release valve device 20. But the plastic tubes 18c should have small inner collars or ridges that can catch securing ridges around the lower ends of the pegs 18a so as to prevent them from ever fully coming out of their tubes 18c (these securing ridges are not shown in any of the drawings).

Also, on the front and rear rims of the diaper device are air-flow vents 16. In other embodiments, they may can be manually closed off or partially closed to control the power of suction created during a flushing or while drying the skin of a user.

There are straps 12 at the top to help hold the diaper together and onto a user. To accomplish this, it may incorporate fasteners, buckles, ties, adhesives, or hook and loop technology. For this first embodiment, I have chosen to hold the diaper onto a user by means of hook and loop technology (best illustrated in FIGS. 1-3, and 5-8). Therefore, the top front side of the diaper has hook patches 36, one on each side, and both sides of the top back end has loop covered straps 12. Soft padded suspenders could be attached and used to further aid in holding the diaper device 10 onto the user if necessary, especially for babies who have such small abdomens and for heavyset or overweight users whose anatomy make it difficult to achieve a good fit.

Last but not least, in terms of the diaper device 10 itself that is worn by a user, there are a variety of pads and cushions 15 inside the cradle shell 22 to provide comfort for the user. These cushions 15 help protect the skin from rubbing back and forth against hard plastic. They should also be greatly water resistant. Upon the diaper seat 54, and under the anus bib 30h is a central seat cushion 15d likely made of neoprene ranging from 1 to 5 mm thick with a water resistant fabric glued on top (or this fabric could be applied to the neoprene rubber as it solidifies during manufacturing) much like the material used for scuba suits and mouse pads. Along the inner walls of the diaper shell 22 is a shell cushion liner pad 15b also made of neoprene 1 to 5 mm thick. There should be some air ventilation channels 15c (divides or gaps) between the central cushion 15d and liner pad 15b and perhaps along the cushions and pads 15 themselves to help naturally circulate air and evaporated moisture from the user to reduce and minimize overheating. Finally, there is a back lumbar cushion 15a and a front abdominal cushion 15e. These two cushions could also be made of neoprene but may also be more padded with synthetic water resistant foam inside. Again, they should be fashioned in such a way as to have some ventilation channels 15c or gaping seams to let air flow out better and to allow heat to escape and sweat to evaporate. Vinyl may be another water resistant material used as part of the final layering of these cushions and pads. Of course, these cushions and pads 15 could also be decorated with printed patterns to illustrate a variety of imagery and design themes like floral patterns, fruits and vegetables, colorful shapes, animals, airplane and naval themes, space travel and astronomy themes, holiday themes, movie themes, nature themes, etc.

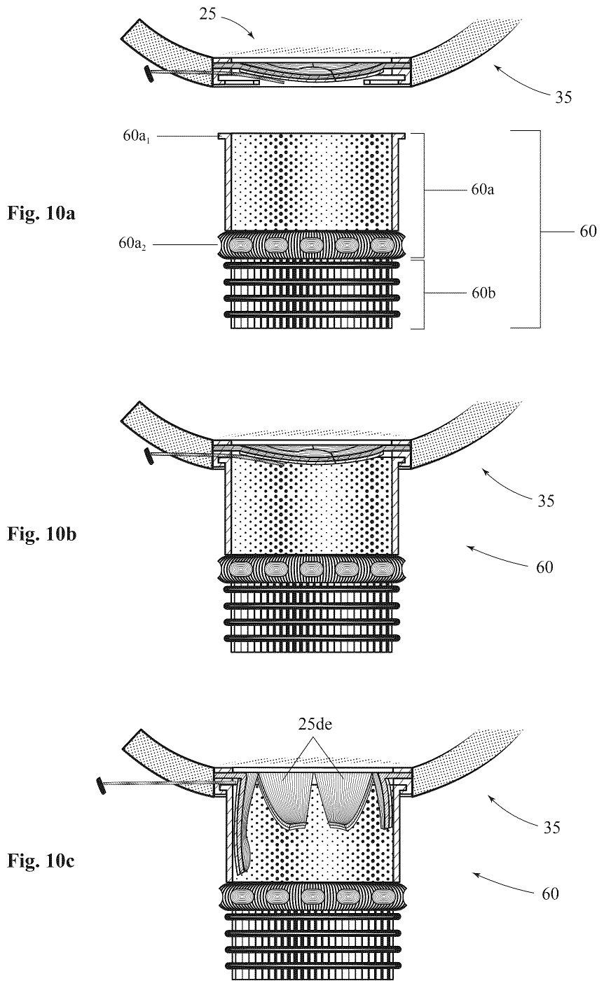

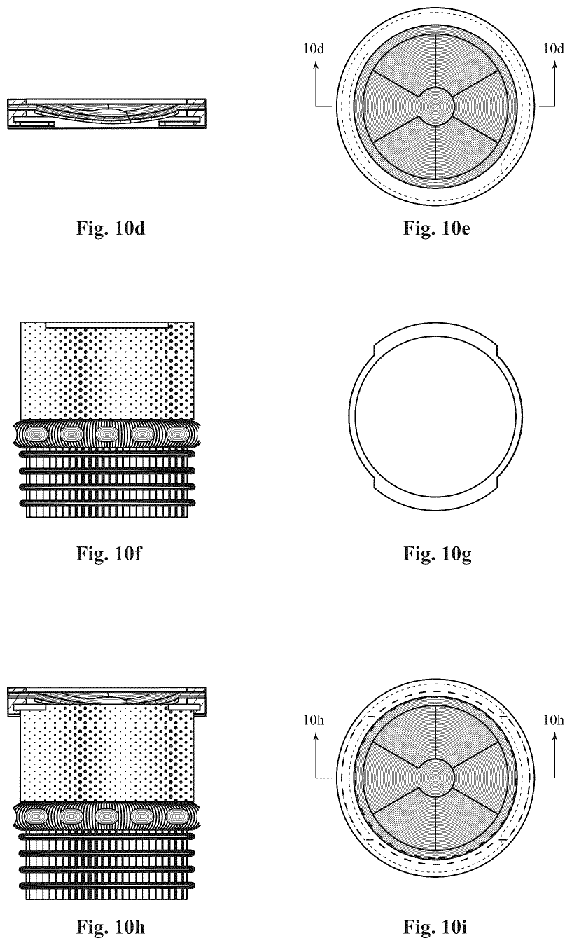

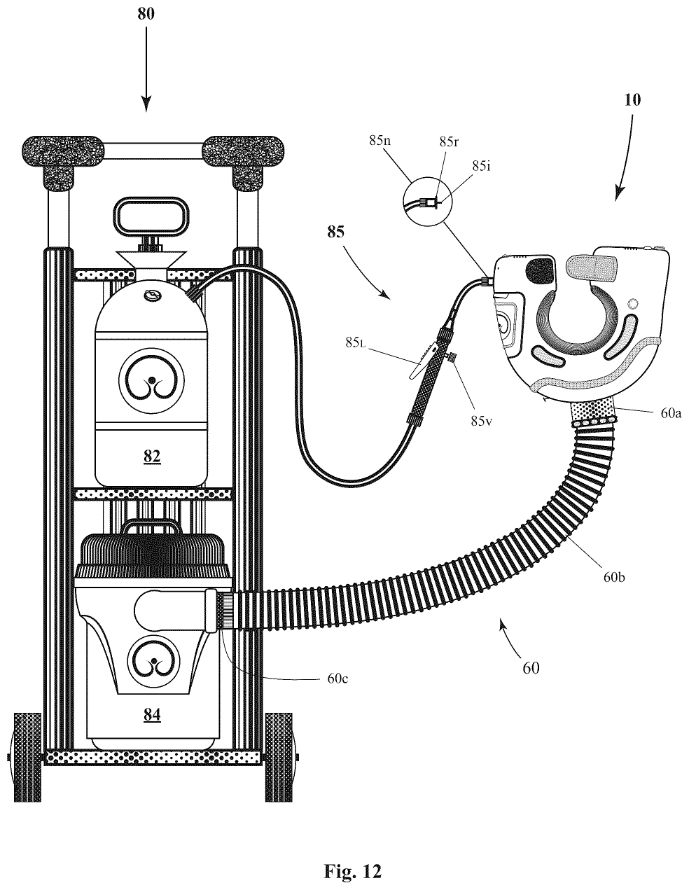

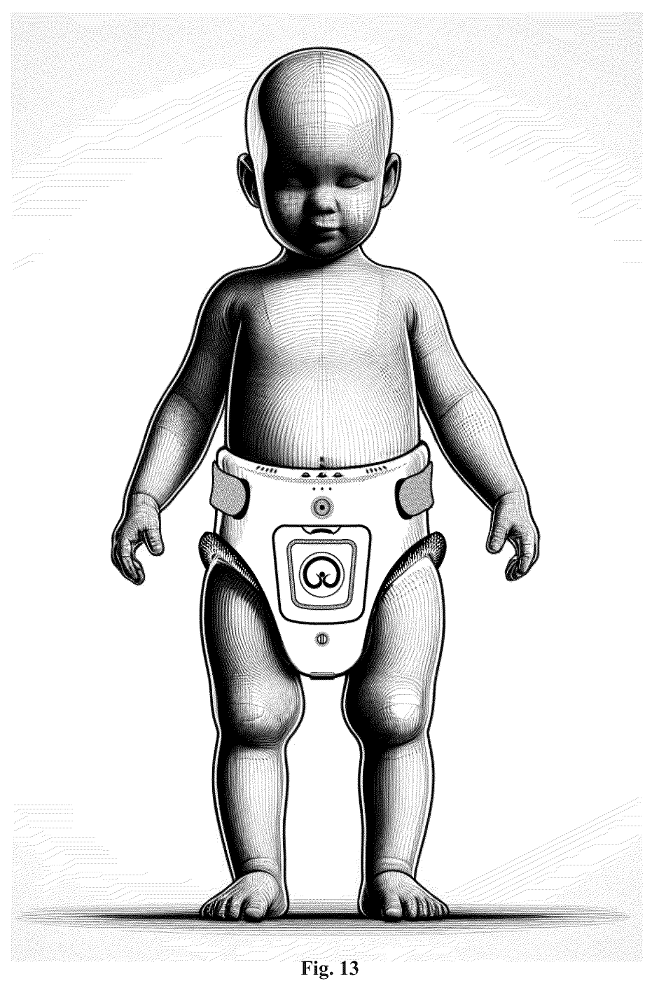

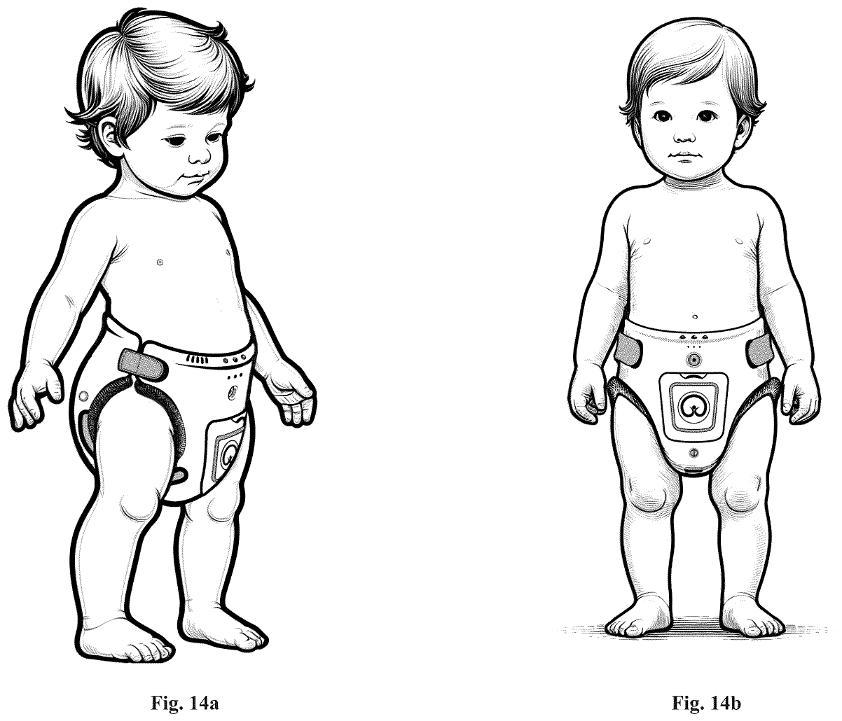

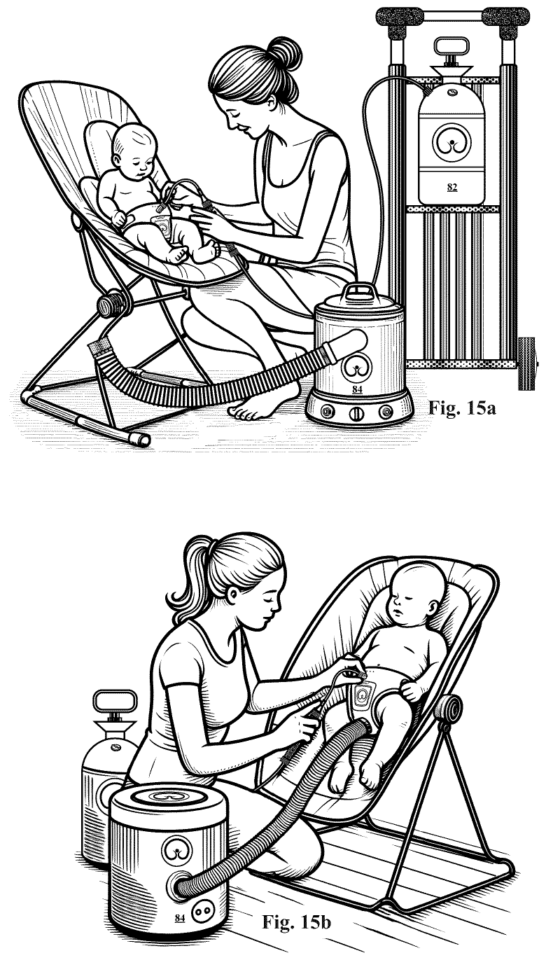

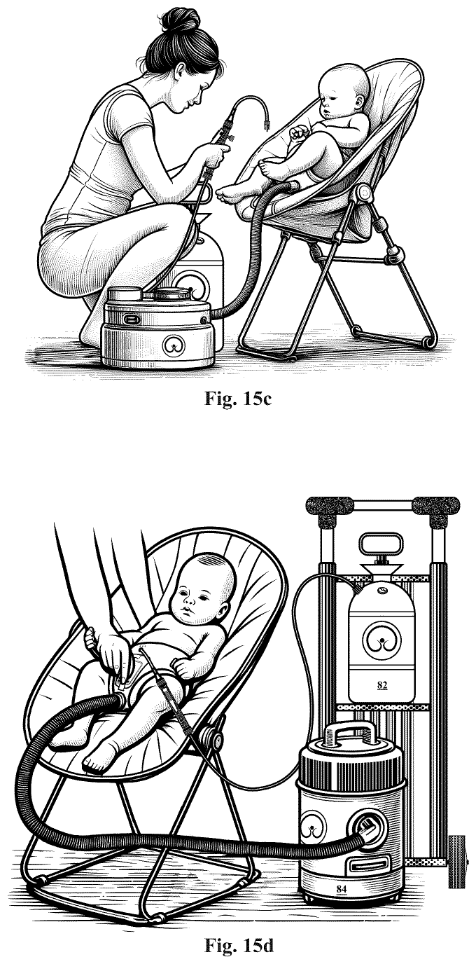

By reviewing FIG. 12, one can get a good idea of the additional components that come with the advanced diaper system, which include a waste drain hose 60 that should be adequately long enough to work with and wide enough to allow human fecal matter to easily flush through, roughly 60 to 100 cm long and 4 to 6 cm in diameter, possibly a little wider for adults or large animals. As diagramed in FIG. 10a, the waste drain hose nozzle 60a (about 3 to 6 cm in length and made of molded plastic) has a finger turn grip 60a2 at its lower base, possibly made of rubber. At the top end there are nozzle securing ridges 60a that extend outward and are used to attach and secure the waste drain hose 60 to the waste chamber drain 25 so that no spilling or leaking of bodily waste occurs during a flush. At the other end of the waste drain hose 60 is a rubber waste drain hose sleeve 60c for a connecting means. It can be better understood by studying FIGS. 2, 6, 10a-10i, and FIG. 12.

Other components and accessories are a flushing hose 85 of adequate size and length (about 1 to 2 cm in diameter). It has a handle and actuator or flow control valve lever 85L to open and close a flow control valve 85v that allows or prohibits the flow of water and a water bottle or tank 82 (size may vary, 3 to 12 liters for home use or smaller such as 200 to 500 ml bottles for travel and portability needs), which can either be pressurized by a built-in hand pump (similar to a multipurpose pressure sprayer that can be purchased at a hardware store or garden supply center) or by an electric water pump like those used in fish aquariums. The flushing hose 85 should come with adapter(s) so that it could be connected to public utility waterlines, instead of using a water storage tank 82, if ever necessary or if preferred. Or a separate longer flushing hose to be connected to utility lines could be included. If for any reason one does not have a water storage tank 82 at the time a diaper flushing is needed, one can do so by connecting it to any public utility waterline in or at the home, such as a sink waterline, toilet waterline, or garden hose water line or any other place with public pluming such as at a restaurant, hospital, rest stop, airplane, or camp site to name a few. The water pressure provided by a public utility waterline should be sufficient enough to flush the diaper device 10 adequately. The other end of the flushing hose, has a flushing hose nozzle 85n and flushing hose nozzle insert 85i that connects into the diaper's navel flushing hose port 50 and inlet 50c using flushing hose nozzle securing ridges 85r.

One other important component is a storage waste tank 84 or waste collection container 84. It can collect, store, and transport about 3 to 12 liters of bodily waste. Smaller storage waste containers could be used for convenience or travel. Each container 84 is to have a pour spout, ideally one that can be turned upward from a stored side position in which case would have a rubber O-ring seal in its swivel joint and which could connect to a waste drain hose 60. The rubber drain hose sleeve 60c on the waste drain hose tube 60b will help provide an adequate seal to prevent spills or leaks of bodily waste or flushing and cleaning fluids.

These additional components and accessories can all be stored and transported by means of a small portable cart 80, which has an extendable handle, two shelves (one for a water tank 82 and one for a waste collection container 84) and two wheels at the bottom. It can be placed in a vertical upright standing position or in a leaning position. These portable carts 80 can be made small enough for travel, about the size of carry-on luggage with handles that can be pushed in for overhead storage on a plane, train, or bus or extended to dolly and move around. These portable carts 80 could also have padded straps so that one could carry one on their back like a back-pack. Another possibility, when it comes to babies, is to design a baby stroller that can store a water tank and waste collection container 84 behind and/or under it, so that it can all be on the go together; the baby, the stroller, the tanks, and hoses. In fact, a parent or caretaker could even have a baby hooked up for flushings while strolling the baby around.

### Detailed Description of the Invention's Function and Operation

Here is a more detailed description of the function and operation of the first embodiment of the diaper device and system, along with the function and operation of its parts.

As already discussed in some detail, this advanced high-tech diaper system is to help manage bodily waste in a safer more sanitary and convenient way. This advanced diaper system is essentially a wearable, flushable bedpan comprising a rigid plastic diaper with a built-in bodily waste collection chamber 23, waste chamber drain valve 25, and a flushing tubes network 70 that may incorporate pressurized water tanks 82, a bodily waste collection container 84, flushing hoses 85, connection adapters, a waste drain hose 60, and a portable cart 80 to transport the system.

A user, such as a baby, an older person, one who is bedridden, or one who is incontinent or hospitalized or mentally challenged, can put one of these diaper devices 10 on or have one put on by a caretaker by flexing the diaper into a more wider open manner. One can fit the user's body into it by holding the front portion with one hand and the back portion with another hand and gently pulling them in opposite direction away from one another. This should provide a large enough gap to slide the diaper up and around a user's genitals, anus, and waist. Then a user or caretaker would use the hook and loop straps 12 and patches 36 to secure the diaper device 10 over the user's groin area and around his or her waist. As mentioned, the diaper comes with padded cushions 15 to help add comfort to the user. The padded cushions 15 could also help to keep the diaper in place and prevent it from sliding down as it becomes full of bodily waste and thus heavier.

A user could then defecate and expel fecal matter through the stool passage port opening 540 and into the waste collection chamber 23 through the available anus saddle seal fissure 30f, passing through the thin rubber anus saddle seal flaps 30e that serve as a one-way check valve, of the anus saddle seal valve 30. Stool could also pass through an adequately sized hole in the anus saddle 30 that could then close back on its own due to the tension in the rubber it's made of if that turns out to work better than an elongated fissure.

A user may also urinate into the available urinal, where a super absorbent sponge 40f absorbs it, causing it to expand and push out the urinal crotch door faceplate 40d and faceplate seal 40b. Therefore, when the urinal sponge 40f is fully saturated, it can clearly be indicated by visual inspection. The urinal sponge guard cage 40g that holds the absorbent sponge 40f in place serves the purpose and function of allowing the sponge 40f to expand in an outward direction only, to keep the male genitals from coming into direct contact with the urinal sponge 40f, and to allow the faceplate 40d to be used as a compressor to push in and compress the urinal sponge 40f, squeezing out urine, before and during a flushing, so that it may drain away the urine into the waste chamber 23. In other words, the sponge 40f can be pressed up against the sponge guard cage 40g and squeezed to push out any fluids. This becomes routine and part of the flushing process. As the diaper 10 is being flushed with water and possibly cleaning fluids, it is necessary to push in the crotch door faceplate 40d and urinal sponge 40f multiple times to squeeze out any absorbed urine from the urinal sponge 40f as it is being sprayed, soaked, and drenched in flushing fluids during a flushing. The user or caretaker should push in the faceplate 40d a few more times after the use of pressurized water and/or cleaning fluids are used to expel any remaining fluids to drain away. This will also help it fully dry sooner.

It should be noted here that during any flushing, air circulates throughout the diaper, due to natural fluid dynamics of the water and surrounding air, given that the diaper has adequate air intake valves and vents as it should. And when the waste collection container 84 is used, it can be equipped with an electric motorized fan that acts as a vacuum, helping to pull the bodily waste contents through the drain 25 and waste drain hose 60. This suction may increase the airflow and drying effect. It's important not to have an electric motor too powerful or the suction too strong (if not enough incoming air from vents and/or valves) or else it could potentially cause injury to the user.

Bodily waste may also be detected and indicated by electronic means, whereby chemical detectors are used along with LED bulbs (48y, 48r, 48o) that light up to communicate to the user or caretaker that the diaper is loaded or full of urine, stool, or gas and needs to be cleaned. Electronic detectors may also trigger warnings that the diaper 10 is soiled or full by mechanical movements like when the waste chamber belly 35 pushes downward or drops at an angle, due to the weight of bodily waste, or when bodily gas or fecal matter presses against the rubber gas pressure indicator bulbs 18d and thus causes the bodily gas indicator pegs 18a to push up, or when the sealed viewports 27L, 27R, 32L, 32R push outward.

When it's time to flush and clean the diaper device 10, one may connect the waste drain hose 60 to the diaper's waste drain valve 25 by unscrewing and removing the waste drain valve lid 25h. And then turning the ridged nozzle 60al of the waste drain hose 60, clockwise, in the groove between the available waste chamber drain securing ridges 25c and plastic rim of the bottom waste chamber drain securing ring 25b.

The other end of the waste drain hose 60 may either be connected to a waste collection container 84, which may or may not have a powered vacuum unit (as in a wet shop-vac) to receive the flushed contents for later disposal or be draped directly over a toilet or appropriate outdoor location.

It should now be safe to use one's fingers to grab the waste chamber drain securing lever handle 25f to pull out the waste chamber drain securing lever 25g, which holds the waste chamber drain 25 in a closed position. At this point, the waste drain flippers 25de will naturally open up by dropping downward, due to gravity and the weight of any bodily waste. If not, the flushing water and cleaning fluids, along with any powered airflow or suction from the waste collection container's 84 vacuum fan, should open them sufficiently for any bodily waste to flush through and out into and down the waste drain hose 60.

Then one may connect the flushing hose 85 that is connected to a water tank 82, which may be filled with warm water for added comfort as well as other cleaning additives and deodorizers, to the navel flushing hose port 50 and inlet 50c, securing it by turning its ridged flushing nozzle 85n clockwise between the flushing port securing ridges 50a and flushing port gasket seal 50b. If the water tank 82 being used is a hand-pump tank, then it may need to be pressurized at this point by pulling and pushing its pump handle up and down a number of times. Warm water and possibly cleaning fluids can be flushed at pressure through a number of acrylic flushing tubes 69 and out one-way check valves 52, 56 to spray the inside of the waste chamber pot 23 and to flush it clean as well as through a bidet 55 tube 69m to spray and clean the anus. This bidet 55 can be shut off when not needed or opened when it is needed using the bidet flow control valve 33. The flushing fluids may then be drained down the waste chamber drain 25 and down the waste drain hose 60 into either a toilet, waste collection container 84, waste receptacle, or appropriate outdoor location.

If and when the waste collection container 84 needs to be emptied, one may use the waste drain hose 60 or not. It should be possible to open the waste collection container's 84 pour spout, swivel it into the best position for pouring, and then pour its contents of collected bodily waste into a toilet, waste receptacle, or appropriate outdoor location. Again, in this embodiment being discussed, one may bypass the waste collection container 84 altogether if necessary by draping the waste drain hose 60 over any toilet or outdoor area like when one is camping at a camp site. A user may also sit on any toilet or outdoor area that is appropriate and open the waste drain valve 25 to flush out and empty any bodily waste.

### OTHER POSSIBLE EMBODIMENTS OF THE INVENTION

A second significant embodiment of this diaper device 10 involves altering the urinal 45 so that it doesn't use a urinal sponge 40f at all and therefore no urinal sponge guard cage 40g either. A user could urinate into a more open urinal 45 without a sponge 40f or sponge cage 40g to drain down the urinal sink 38 and into the waste chamber. It could be lined with silicone that would lead into the urinal sink 38 and urinal drain tube, creating a single continuous seal and provide added comfort for male genitals

A third quite notable embodiment of this diaper invention is one where urine can be first collected in a smaller chamber or container rather than the main waste chamber 23. This helps to avoid any possible problems of the fecal matter becoming overly saturated with urine and sloshing and splattering or spilling back through the anus fissure 30f, especially while the user is lying down. The first embodiment and others have been designed to likely combat such issues, but it still may prove advantageous to keep the main waste chamber pot 23 as dry as possible during use. Anti-slosh baffles could also be incorporated into the main waste chamber 23. This smaller urinal chamber would be within the walls of the diaper shell (between 22i and 22e). Ideally, the waste chamber belly 35 could serve as this chamber, since the flushing tubes 69 and one-way check valves 52, 56 are not located in this area. The only obstacle would be the waste drain valve 25. But a plastic urinal collection bag like those commonly used in hospitals for catheters known as “leg-bags” and made of polyvinyl chloride or ethylene vinyl acetate could be designed and shaped somewhat like a horseshoe to be placed around the waste drain 25. It may also be possible to not use a urinal collection bag but strictly the plastic walls of the diaper shell 22 of the waste chamber belly 35 as a closed container. If then, it would have an air outlet hole or tube to let air empty out as urine fills it up. This urine collection container (also the waste chamber belly 35) could also have an overflow tube that runs from it into the main waste chamber 23 so that urine could flow out into the main waste chamber 23 if the smaller urine collection container 35 becomes full. For this embodiment, the waste chamber belly 35 would either have a urinal drain port 35u of its own near the main waste drain valve 25 as seen in the bottom view of FIG. 8 or the waste drain valve 25 could be designed to allow urine from the smaller urine collection container 35 to drain out of it during a flushing.

Another possible embodiment of this diaper device may have the upper front section, which includes the entire urinal 45 and even the upper back section of the diaper, to be capable of bending outward and down a great deal independently of the waste chamber section 23. Those upper sections would be connected in such a way as to be jointed so that those upper sections can freely move back and forth or if bent downward could spring back into their original position by means of springs, ligaments, or rubber seal tension. These upper sections could be entirely removable as well, so that all one is dealing with at first is the waste chamber 23, diaper seat 54, and anus saddle 30 portion of the diaper, much like a sophisticated bedpan. At times, such as in hospitals when dealing with bedridden patients, the upper portions may simply be in the way, especially if being used strictly as a bedpan. It may not be necessary for a user to actually wear the device. But if they are needed and if the device is to be worn, then the front and back portions could be put in place. This version is a more compounded and jointed diaper device, using additional rubber seals and possibly rubber bands, rubber cords, metal or plastic springs and/or plastic ligaments.

In yet another embodiment of this diaper device 10 of this diaper system, it may be advantageous to make the abdominal front cushion 15e as well as the back lumbar cushion 15a partly or fully inflatable. A separate hand pump could be connected and used to inflate the cushions to the point of proper snugness and fit, similar to how a blood pressure cuff works around a patient's arm. It may also be possible to have a small pump built into the diaper device 10 itself, even as small as a finger pump for the finger or thumb to actuate and pump air into the abdominal and lumbar cushions 15e, 15a. Another option is a small tube that one could attach to the cushions to blow air into them until they reach the desired inflation and fit around the user. These tubes could be small and already attached to the cushions and able to be easily tucked away or pushed inwards into the cushions to be stowed.

An additional embodiment of my diaper invention would be one that doesn't use water or any other liquids to flush it clean but instead uses dry substances like mineral powders and granules, grains, sawdust, clay, sand, dirt, ash, wood shavings, flower petals, pulp, leaves, grass, seaweed, peat moss, etc., thus referred to as a “dry flush” or compost version. Of course many of the embodiments already discussed could serve as both a wet and dry flushing diaper device 10 with some slight modifications, such as designing the waste chamber belly 35 to also serve as a larger lid to the waste chamber 23 that could be opened and closed by means of a hinge or fully removed and then put back on. Then, if one is environmentally conscious and prefers not to use water or they don't have access to water, such as while camping, they could have the choice and option to use dry substances instead. They would need to prep the diaper by turning the diaper upside down, opening the waste drain valve 25 or larger waste chamber belly 35, and pouring in a dry substance of their choice into the waste chamber pot 23, and then of course re-securing the waste drain valve 25 with the securing lever 25g and lid 25h or closing the entire waste chamber belly 35 that was opened like a lid. Once the dry contents become soiled with bodily waste to the point that it needs to be cleaned out, say after a day's use, then it could be opened and emptied much the way one cleans out a kitty litter pan.

A similar option and embodiment to the previous one is an alternate dry flush version that uses plastic or wax coated paper bags or possibly even washable synthetic or cloth fabric bags instead of dry substances as listed above in the first dry flush and compost version. A user or caretaker would prep the diaper by lining the inside waste chamber pot 23 of the diaper with a water proof bag of some sort and possibly pulling the top of the bag through the anus saddle 30 fissure or hole and spreading it over the anus saddle 30. These bags could be made and sold with the advanced hi-tech diaper system or could be purchased separately. Many common multipurpose plastic or wax coated paper bags sold on the market for home use could work. When the diaper becomes full of bodily waste, the diaper can be emptied by opening or removing the waste chamber belly 35, which again serves as a lid in this embodiment, and carefully removing the bag and disposing of it as one would a disposable diaper. This diaper could even be designed so that the diaper seat 54 (also referred to as the waste chamber cover shield) could be opened from the top, to place in clean unused bags and to remove used bags of bodily waste. If this version did not have a separate urine collection container (like the waste chamber belly 35), to collect most of the user's urine, then it might be necessary to add at least some dry substances into the bag placed within the waste chamber pot 23 to absorb any urine draining into it.

An eighth embodiment of this diaper system invention is one that uses a semi rigid diaper device instead of a rigid one. The plastics used to shape the diaper cradle shell 22 could be even more flexible than the cradle shell 22 of the first embodiment and could incorporate portions of the diaper that are very non-rigid, such as the diaper seat/waste chamber cover 54. Instead of being made of rigid or semi-rigid plastic, the diaper seat 54 could be made of silicone rubber or neoprene as well as a woven mesh or net of synthetic straps to hold the weight of the user in a sling or hammock fashion. The rigid portions of the shell 22 could be greatly reduced to a minimum. The continuous surface of the diaper shell 22 would only be enough to serve as a support frame, while much of the diaper shell 22 surface that was rigid in the first embodiment is now instead made of silicone rubber, neoprene, thin vinyl, or padded waterproof synthetic woven fabric, or any combination of these flexible, bendable materials.

A ninth embodiment of this diaper system is one that incorporates a fully non-rigid diaper device, in terms of its cradle shell 22 and diaper seat 54. Of course diaper parts like the waste drain 25, bidet knob 33, bodily gas indicators 18, gas release valve device 20, and all the one-way check valves 52, 52m, 56, 56m, etc. would still include rigid materials. This version of the diaper would have a shell and waste chamber that is almost fully comprised of flexible materials like natural or synthetic rubbers, such as latex rubber, nitrile, silicone, neoprene, or other materials like foam-padded vinyl. This version would need an anus saddle 30 that serves as a very effective non-return valve so as to prevent any backflow of liquid or solid waste. Otherwise, bodily waste could push back through the anus valve 30 when a user applies force onto the flexible, non-rigid shell, especially the bottom of the diaper like when a user sits down or lies down and applies their weight onto the diaper. Another important feature this embodiment may need to include is some sort of minimal rigid undercarriage support to provide at least some distance and volume between the anus saddle 30 and the inner shell surface 22i.

Yet another version or embodiment is a diaper that has no waste chamber belly 35 that can independently move outward or downward to increase the volume of the waste chamber 23. It may not be necessary or at least this version may be suitable for many less extreme uses.

One more alternate embodiment I would like to discuss is a diaper as described in the first embodiment, but one that incorporates a soft flexible rectal insert. It would be a flexible shaft, tube, or conduit made of latex rubber, nitrile, or silicone that could be attached to the stool passage port opening 540 of the diaper seat 54 or could be a part of the anus saddle 30 or entirely separate from the anus saddle 30, yet attached and secured to the anus saddle 30. It would allow feces to be excreted directly from the user's rectum, down the inserted tube, and into the waste chamber 23 in a more sanitary way. This version allows the user to remain very clean by not having their outer skin of their anus or buttocks come into contact with any fecal matter. This way, their anus doesn't have to be showered clean by the built-in bidet 55 and then air dried by the air-drying device and system 75. A user or caretaker would have to insert this flexible rubber tube up into a user's rectum, while first putting the diaper device on, and then activate the inserted top end of the rectal shaft to mechanically expand or blossom open much like a flower or an umbrella to help secure it in place like a catheter and to widen the aperture for stool needing to pass through. However, testing would need to be done to ensure that long term use of such a tube wouldn't cause any harm or injury. This added rectal insert may prove to be impractical and unnecessary.

Although this invention has been discussed in some detail using certain embodiments, it should be known that they are only examples of some of the possibilities. It should further be understood that several changes and combinations could be made for additional embodiments in terms of its use, parts, arrangement, materials, and method of construction without deviating from the protected idea, concept, and scope of this invention that is defined within the claims and, therefore, should not be limited to the description and illustrative drawings, herein.

1. An advanced diaper device with a built-in flushing system, comprising:
(a) water resistant materials that may include but are not limited to polymers like polyethylene, polyvinyl chloride, nylon, resin, acrylic, and other plastics that can be molded into shape as a rigid, semi-rigid, and non-rigid form;
(b) a waste chamber or compartment with a plurality of openings, such as a stool passage port opening on its top surface, a drain opening at the bottom, and smaller holes for one-way check valves to be installed as well as a bodily gas release flue, which will capture and collect and temporarily store bodily waste like urine, feces, menstrual blood, semen, and vaginal discharge;
(c) a urinal or urine compartment that will capture, collect, and temporarily hold bodily waste, such as urine, menstrual blood, semen, and vaginal discharge, which can drain and be emptied into said waste chamber or into a urine collection container of its own;
(d) a waste drain valve installed at the bottom of said diaper device, which is also the bottom of said waste chamber, that can be opened and adequately closed to prevent any significant amounts of bodily waste leakage or spillage and which will allow said bodily waste, previously collected and stored, to be flushed out and disposed;
(e) a bodily waste drain hose that has connecting means at one end to securely fit and connect to said diaper's said waste chamber drain and the opposite end can optionally securely fit into most common wet-shop vacs by connecting means or be draped over any collection container or toilet or appropriate disposal reservoir or outdoor area;
(f) a flushing hose port incorporating a waterproof gasket seal and a connecting means so that a flexible flushing hose can be securely connected to said diaper device to provide flushing cleaning fluids into said diaper device;
(g) a network of tubes or conduits that allow cleaning fluids to enter and exit said waste chamber and said urine compartment of said diaper device so that said bodily waste can be adequately flushed out;
(h) a plurality of one-way check valves connected to said network of tubes and installed into said waste chamber and said urinal to allow cleaning fluids, air, and bodily waste to enter and exit said diaper device in one direction but not the opposite direction, whereby said diaper device can be adequately flushed out and cleaned;
(i) a flexible flushing hose with connecting means at one end to securely fit inside and connect to said diaper's flushing hose port, while the opposite end has connecting means to securely connect to a water tank and/or waterline;
(j) a plurality of water resistant seals made of flexible, waterproof materials that may include but are not limited to elastic rubbers like silicone, neoprene, nitrile, and latex made from natural tree rubber, elastomers, and/or flexible, water resistant materials like polymer foam, vinyl, and cork that come into contact with the user's skin as well as other parts of said diaper device to help divide said diaper device into compartments and chambers to prevent or adequately reduce leakage or undesired cross contamination;
whereby said diaper device is able to be used in cooperation with other common devices and means like waste collection containers, with or without vacuum power means, portable water tanks, and water supply lines to flush out bodily waste and thus is flushable and cleanable and can be reused repeatedly.

2. The advanced diaper device in claim 1, wherein said diaper device is non-disposable as well as cleanable to the point it can be used repeatedly multiple times and therefore is reusable.

3. The advanced diaper device in claim 1 further includes an anus saddle seal valve made of an elastomer with a soft tacky adhesive texture that is both a waterproof seal and a one-way check valve due to its rubber membrane flaps that come together and then fold down into the said waste chamber, which fits into the stool passage port opening to provide an adequate seal along a user's skin around his or her anus, so that bodily waste cannot spill or leak beyond said waste chamber nor said anus saddle seal valve.

4. The anus saddle seal valve in claim 3 further includes a mini air-flow one-way check valve within the anus saddle seal, whereby air, possibly warmed up by battery-powered heating coils, can flow into the anus saddle seal to help dry the skin of a user and provide needed air flow to empty waste contents collected in said waste chamber.

5. The anus saddle seal valve in claim 3, wherein the diameter or maximum length of the opening is 6.5 cm or smaller to maximize protection for the skin of a user against coming in contact with bodily waste, while at the same time being sufficiently large enough to allow fecal matter to adequately pass through and into the waste collection chamber.

6. The advanced diaper device in claim 1 further includes a bidet system comprising a mini flushing tube connected to said flushing tubes network with a flow control valve and a mini one-way check valve at its opposite end that is installed into said anus saddle seal valve to clean a user's anus with water and/or other cleaning fluids by streaming and/or spraying fluid means of said mini one-way check valve.

7. The advanced diaper device in claim 1 further includes water absorbent materials, such as but not limited to super absorbent sponges placed within the urinal to initially absorb urine and menstrual blood before being drained into said waste chamber and to wick it away from the skin and genitals of a user, absorbent pads that can be placed within said urinal and around said anus saddle seal valve, which can be cleaned and reused or disposable, and possibly the use of super absorbent polymers or absorbent gel materials like sodium polyacralate crystals.

8. The advanced diaper device in claim 1, wherein said diaper device is primarily, though not necessarily entirely, a rigid plastic structure, whereby its diaper cradle shell and inner waste collecting chamber does not easily bend or flex, thus preventing bodily waste from being improperly pushed or squeezed back out of said anus saddle seal valve, such as when a user applies his or her own weight onto said diaper device like while sitting down.

9. The advanced diaper device in claim 1, wherein said diaper device is primarily, though not necessarily entirely, a non-rigid structure, whereby most of its cradle shell and inner waste collection chamber is pliable and flexible, in which case incorporates an even more effective one-way anus saddle seal valve, so that feces and other bodily waste cannot backup through said anus saddle seal valve if a user has pressure applied to said diaper shell like when bumping into walls, furniture, or when sitting or lying down.

10. The advanced diaper device in claim 1, wherein said waste chamber has a moveable hatch-like waste chamber belly built into the bottom of the diaper's cradle shell and attached by an expandable rubber seal all around its circumference so that it can drop down to increase the volume of said waste chamber as it becomes filled with bodily waste and may be fully removed from said diaper device, if ever necessary, and then reattached and could double as a urine collection container to collect urine being drained from the urinal.

11. The advanced diaper device in claim 1 further includes bodily waste indicating means, comprising of moveable rods or pegs that rise and fall within pressure tubes due to an increase or decrease in solid or liquid bodily waste as well as bodily gas pressure within said waste chamber, viewports installed into said cradle shell for visual inspections and which may expand outward due to increased bodily waste in the form of solids, liquids, and/or gas pressure, LED lights that can light up to indicate when said diaper device is full or nearly full and needs to be flushed, and/or auditory alarm devices that beep or chime to let caretakers know the diaper has been soiled or used and may need to be flushed.

12. The advanced diaper device in claim 1 further includes inner padding comprising an inner rubber lining made of silicone or neoprene, synthetic foam padding, as well as soft water resistant and/or water absorbing fabrics to add comfort to the user's buttocks and skin that come in direct contact with said diaper device to serve as a cushion.

13. The advanced diaper in claim 1, whereby its said shell cradle is compounded and jointed so that its upper front and upper back portions can greatly swivel, back and forth, towards and away from a user to make it easier to put on and is entirely removable so that said diaper device can more easily be used as a bedpan if needed and preferred.

14. The advanced diaper device in claim 1 incorporates bodily waste detection means, such as electronic detection devices to detect certain odors related to bodily waste or chemically activated materials that can be used to detect when bodily waste is present like a material that changes color when it comes into contact with urine or bodily gas.

15. A bodily waste management system that provides a means for capturing, collecting, storing, transporting, flushing, and disposing bodily waste, comprising:
(a) a diaper device comprising a built-in flushing tubes network, one-way check valves, water resistant seals, bodily waste collection chamber, waste chamber drain valve that can be opened and closed, urinal, and a flushing hose port, which can be worn by a user or used as a bedpan,
(b) a portable container or water tank that is to be filled with water and optionally other cleaning fluids or additives, which produces a flushing flow of liquid into said diaper device through a flexible hose either by the aid of gravity, manually or electrically powered pumps, external vacuum forces, or a combination thereof, whereby bodily excrement can be flushed out of said waste chamber through said waste chamber drain valve of said diaper device,
(c) a flexible water hose with a flow control valve that connects to a flushing hose port in said diaper device at one end, while the other end connects to the aforementioned portable water tank, or at times a water supply line, so that water and other cleaning fluids can be flushed through said flushing tubes network, said one-way check valves, said urinal, and said waste chamber to empty and clean said diaper device,
(d) a flexible hose sufficiently large enough in diameter to allow fecal matter to pass through, which connects to said diaper device so that bodily excrement can be drained and transferred to a storage container, toilet, sewer line, or outdoors,
(e) a portable waste collection and storage container that hooks up to said diaper device by means of said flexible waste drain hose to receive all flushing contents, such as water, additives, bodily excrements, and so forth for the purpose of storing and disposing of said flushing contents flushed from said diaper device.

16. The bodily waste management system in claim 15, wherein said cleaning fluids are pumped from said portable water tank into said diaper device by means of a hand operated pump that can build up sufficient pressure within said portable water tank.

17. The bodily waste management system in claim 15, wherein said cleaning fluids are pumped from said portable water tank into said diaper device by means of an electrically powered pump.

18. The bodily waste management system in claim 15, wherein said bodily waste collection container includes an electrically powered vacuum pump, which also provides a means of air circulation to help dry the skin of a user during and after a flushing.

19. The bodily waste management system in claim 15, wherein said diaper device uses waste collection bags that may be but not limited to materials that are water resistant, biodegradable, washable cloth, synthetic, disposable or non-disposable, which is to be placed inside said waste chamber with its top pulled through and draped over the anus saddle seal valve and used to capture and collect said bodily waste when water and cleaning fluids are not available or desirable and to be properly disposed of the way current traditional disposable diapers are disposed of and may use particulates of matter inside to help absorb fluids and to serve as deodorizers or as a compost alternative, which may include but are not limited to sawdust, cellulose, powdered minerals, soil, and so forth, whereby said waste is adequately cleaned out into said collection container.

20. A method for managing bodily waste, comprising the steps of:
(a) providing a diaper device comprising a built-in flushing tubes network, one-way check valves, water resistant seals, bodily waste collection chamber, waste chamber drain valve that can be opened and closed, urinal, and a flushing hose port, which can be worn by a user or used as a bedpan,
(b) providing a portable container or water tank that is to be filled with water and optionally other cleaning fluids or additives, which produces a flushing flow of liquid into said diaper device through a flexible hose either by the aid of gravity, manually or electrically powered pumps, external vacuum forces, or a combination thereof, whereby bodily excrement can be flushed out of said waste chamber through said waste chamber drain of said diaper device,
(c) providing a flexible water hose with a flow control valve that connects to a flushing hose port in said diaper device at one end, while the other end connects to the aforementioned portable water tank, or at times a water supply line, so that water and other cleaning fluids can be flushed through said network of tubes, said one-way check valves, said urinal, and said waste chamber to empty and clean said diaper device,
(d) providing a flexible hose sufficiently large enough in diameter to allow fecal matter to pass through, which connects to said diaper device so that bodily excrement can be drained and transferred to a storage container, toilet, sewer line, or outdoors,
(e) providing a portable waste collection and storage container that hooks up to said diaper device by means of said flexible waste drain hose to receive all flushing contents, such as water, additives, bodily excrements, and so forth for the purpose of storing and disposing of said flushing contents flushed from said diaper device,
(f) capturing said bodily waste by means of said bodily waste chamber of said diaper device,
(g) collecting said bodily waste using said bodily waste chamber of said diaper device,
(h) storing said bodily waste by means of said bodily waste chamber of said diaper device if necessary until said bodily waste can be flushed out and disposed of,
(i) flushing and clearing out said bodily waste, along with any cleaning fluids, from said waste chamber of said diaper device by means of said flushing tubes network, said one-way check valves, and said waste chamber drain, whereby said bodily waste can be dislodged and emptied into a toilet or said portable waste collection and storage container by means of said waste chamber drain hose that is connected to said waste chamber drain valve,
(j) disposing the collected bodily waste from said waste collection container by pouring and emptying said collected bodily waste into a toilet, sewer line, or appropriate outdoors location, whereby said collected bodily waste is captured, collected, stored if necessary, flushed and cleaned out of said diaper device, and then disposed of adequately and properly.
